(19) United States
(12) Patent Application Publication
(10) Pub. No.: US 2009/0005845 A1
(43) Pub. Date: Jan. 1, 2009

(54) Intra-Atrial parasympathetic stimulation

(76) Inventors: Tamir Ben David, Tel Aviv (IL); Omry Ben Ezra, Tel Aviv (IL); Ehud Cohen, Ganei Tivka (IL)

Correspondence Address: COOPER & DUNHAM, LLP, 1185 AVENUE OF THE AMERICAS, NEW YORK, NY, 10036, US

(21) Appl. No.: 12/012,366

(22) Filed: Feb. 1, 2008

Related U.S. Application Data
(60) Provisional application No. 60/937,351, filed on Jun. 26, 2007.
(60) Provisional application No. 60/965,731, filed on Aug. 21, 2007.

(51) Int. Cl. A61N 1/05

(52) U.S. Cl. 607/122

(57) ABSTRACT

A method is provided, including implanting in an atrial wall of a subject, from within an atrium, a first electrode contact in a vicinity of a parasympathetic epicardial fat pad of the subject, and implanting a second electrode contact in a body of the subject outside of a heart and a circulatory system. A current is driven between the first and second electrode contacts, and configured to cause parasympathetic activation of the fat pad. Other embodiments are also described.

### CROSS-REFERENCES TO RELATED APPLICATIONS

The present patent application claims the benefit of U.S. Provisional Application 60/937,351, filed Jun. 26, 2007, entitled, “Intra-atrial parasympathetic stimulation,” and U.S. Provisional Application 60/965,731, filed Aug. 21, 2007, entitled, “Intra-atrial parasympathetic stimulation,” both of which are assigned to the assignee of the present application and are incorporated herein by reference.

### FIELD OF THE INVENTION

The present invention relates generally to treating patients by application of electrical signals to selected tissue, and specifically to methods and apparatus for applying parasympathetic stimulation.

### BACKGROUND OF THE INVENTION

The use of nerve stimulation for treating and controlling a variety of medical, psychiatric, and neurological disorders has experienced significant growth over the last several decades, including for treatment of heart conditions. In particular, stimulation of the vagus nerve (the tenth cranial nerve, and part of the parasympathetic nervous system) has been the subject of considerable research. The vagus nerve is composed of somatic and visceral afferents (inward conducting nerve fibers, which convey impulses toward the brain) and efferents (outward conducting nerve fibers, which convey impulses to an effector to regulate activity such as muscle contraction or glandular secretion).

The rate of the heart is restrained in part by parasympathetic stimulation from the right and left vagus nerves. Low vagal nerve activity is considered to be related to various arrhythmias, including tachycardia, ventricular accelerated rhythm, and rapid atrial fibrillation. Stimulation of the vagus nerve has been proposed as a method for treating various heart conditions, including atrial fibrillation and heart failure. By artificially stimulating the vagus nerves, it is possible to slow the heart, allowing the heart to more completely relax and the ventricles to experience increased filling. With larger diastolic volumes, the heart may beat more efficiently because it may expend less energy to overcome the myocardial viscosity and elastic forces of the heart with each beat.

Atrial fibrillation is a condition in which the atria of the heart fail to continuously contract in synchrony with the ventricles of the heart. During fibrillation, the atria undergo rapid and unorganized electrical depolarization, so that no contractile force is produced. The ventricles, which normally receive contraction signals from the atria (through the atrioventricular (AV) node), are inundated with signals, typically resulting in a rapid and irregular ventricular rate. Because of this rapid and irregular rate, the patient suffers from reduced cardiac output, a feeling of palpitations, and/or increased risk of thromboembolic events.

Current therapy for atrial fibrillation includes cardioversion and rate control. Cardioversion is the conversion of the abnormal atrial rhythm into normal sinus rhythm. This conversion is generally achieved pharmacologically or electrically. An atrial defibrillator applies an electrical shock when an episode of arrhythmia is detected. Such a device has not shown widespread clinical applicability because of the pain that is often associated with such electrical shocks. Atrial override pacing (the delivery of rapid atrial pacing to override abnormal atrial rhythms) has not shown sufficient clinical benefit to justify clinical use. Rate control therapy is used to control the ventricular rate, while allowing the atria to continue fibrillation. This is generally achieved by slowing the conduction of signals through the AV node from the atria to the ventricles.

Current treatment techniques have generally not demonstrated long-term efficacy in preventing the recurrence of episodes of atrial fibrillation. Because of the high frequency of recurrences (up to several times each day), and a lack of effective preventive measures, many patients live in a constant state of atrial arrhythmia, which is associated with increased morbidity and mortality.

An article by Vincenzi et al., entitled, “Release of autonomic mediators in cardiac tissue by direct subthreshold electrical stimulation,” J Pharmacol Exp Ther. 1963 August; 141:185-94, which is incorporated herein by reference, describes subthreshold electrical stimuli for myocardial excitation. Such excitation was described as being effective in causing the release of autonomic mediators in several types of cardiac tissue derived from rabbit, guinea pig, dog, and cat.

U.S. Pat. No. 5,411,531 to Hill et al., which is incorporated herein by reference, describes a device for controlling the duration of A-V conduction intervals in the heart. Stimulation of the AV nodal fat pad is employed to maintain the durations of the A-V conduction intervals within a desired interval range, which may vary as a function of sensed heart rate or other physiologic parameter. AV nodal fat pad stimulation may also be triggered in response to defined heart rhythms such as a rapid rate or the occurrence of premature ventricular depolarizations (PVCs), to terminate or prevent induction of arrhythmias.

Cooper T B et al., in “Neural effects on sinus rate and atrioventricular conduction produced by electrical stimulation from a transvenous electrode catheter in the canine right pulmonary artery,” Circulation Research 46:48-57 (1980), which is incorporated herein by reference, studied the effects on sinus rate and atrioventricular (AV) conduction of electrical stimulation from a 12-polar electrode catheter advanced into the right pulmonary artery of 21 anesthetized dogs. In each experiment, the distal tip of the electrode catheter was positioned at a standard fluoroscopic site, and a sequence of bipolar electrograms was recorded during sinus rhythm from the 11 adjacent catheter electrode pairs using a standardized technique. Stimulus-strength response testing was performed from each catheter electrode pair during spontaneous sinus rhythm and during atrial fibrillation sustained by rapid atrial pacing. Negative chronotropic and negative dromotropic effects persisted throughout 5-minute periods of stimulation from the optimal stimulation site and could be modulated by varying stimulus parameters. Using neurophysiological and neuropharmacological techniques, they demonstrated that these effects were produced by stimulation of preganglionic parasympathetic efferent nerve fibers.

Quan K J et al., in “Endocardial Stimulation of Efferent Parasympathetic Nerves to the Atrioventricular Node in Humans: Optimal Stimulation Sites and the Effects of Digoxin,” Journal of Interventional Cardiac Electrophysiology 5:145-152 (2001), which is incorporated herein by reference, describe a study to identify optimal sites of stimulation of efferent parasympathetic nerve fibers to the human atrioventricular node via an endocardial catheter and to investigate the interaction between digoxin and vagal activation at the end organ.

Bluemel K M et al., in “Parasympathetic postganglionic pathways to the sinoatrial node,” Am J Physiol 259(5 Pt 2):H1504-10 (1990), which is incorporated herein by reference, describes the mapping of the ventral epicardial surface of the right atrium in dogs. A concentric bipolar exploring electrode was used to stimulate (during the atrial refractory period and using trains of five to eight stimuli per beat) systematically in the epicardial regions between the right pulmonary vein complex and the SA node. The authors report that the primary vagal postganglionic pathways to the SA nodal region are subepicardial and adjacent to the SA node artery along the sulcus terminalis.

U.S. Pat. No. 6,298,268 to Ben-Haim et al., which is incorporated herein by reference, describes apparatus for modifying cardiac output of the heart of a subject, including one or more sensors which sense signals responsive to cardiac activity, and a stimulation probe including one or more stimulation electrodes which apply non-excitatory stimulation pulses to a cardiac muscle segment. Signal generation circuitry is coupled to the one or more sensors and the stimulation probe. The circuitry receives the signals from the one or more sensors and generates the non-excitatory stimulation pulses responsive to the signals.

U.S. Pat. No. 6,292,695 to Webster, Jr. et al., which is incorporated herein by reference, describes a method of controlling cardiac fibrillation, tachycardia, or cardiac arrhythmia by the use of an electrophysiology catheter having a tip section that contains at least one stimulating electrode, the electrode being stably placed at a selected intravascular location. The electrode is connected to a stimulating means, and stimulation is applied across the wall of the vessel, transvascularly, to a sympathetic or parasympathetic nerve that innervates the heart at a strength sufficient to depolarize the nerve and effect the control of the heart.

US Statutory Invention Registration H1,905 to Hill, which is incorporated herein by reference, describes an endocardial pacing and/or cardioversion/defibrillation lead having a plurality of electrodes and a mechanism for adjusting the exposed surface area of one or more electrode and/or the position and/or angular orientation of an electrode along a lead body. In an embodiment, movable electrodes may be positioned to facilitate delivery of electrical stimulation through the atrial wall or the superior vena cava wall to autonomic nerves to influence sinus heart rate, the A-V interval, and blood pressure or the like. For example, vagal nerve stimulation may be effected through the atrial wall by an electrode that is oriented towards the vagal nerves. The vagal stimulation may be delivered during an episode of atrial fibrillation or tachycardia in order to slow the ventricular heart rate response to the atrial heart rate.

U.S. Pat. No. 7,269,457 to Shafer et al., which is incorporated herein by reference, describes a medical procedure including stimulation of a patient's heart while stimulating a nerve of the patient in order to modulate the patient's inflammatory process. More particularly, the medical procedure includes pacing the ventricles of the patient's heart while stimulating the vagal nerve of the patient.

U.S. Pat. No. 6,937,897 to Min et al., which is incorporated herein by reference, describes an electrical lead equipped with cathode and anode active succession electrodes for positioning in the vicinity of the His bundle tissue. The lead includes a lead body for carrying conductors coupled between electrodes located at or near the distal lead end and a connector assembly located at the proximal lead end for connecting to an implantable pacemaker. The electrode is shaped, at the distal end, for positioning and attachment in the His bundle and branches thereof, cathode and anode electrodes co-extensive with the lead body. The cathode and anode electrodes may be helical screw-in type or equivalent electrodes adapted for secure fixation deep within the His bundle tissue or the tissue in the vicinity of the His bundle.

PCT Publication WO 02/22206 to Lee, which is incorporated herein by reference, describes a pacing lead characterized by a screw-in tip that is longer than conventional tips and is provided with an electrically active distal electrode, which is insulated from the proximal part of the screw tip of the pacemaker lead. This electrically active distal screw-in tip is extended from the right ventricular septal endocardium into the left side of the interventricular septum and is used for left ventricular pacing with optional properly synchronized right ventricular pacing.

U.S. Pat. No. 6,611,713 to Schauerte, which is incorporated herein by reference, describes an implantable device for diagnosing and distinguishing supraventricular and ventricular tachycardias includes electrodes for stimulating parasympathetic nerves of the atrioventricular and/or sinus node; electrodes for stimulating the atria and ventricles and/or for ventricular cardioversion/defibrillation; a device for producing electrical parasympathetic stimulation pulses passed to the electrodes; a device for detecting the atrial and/or ventricular rate, by ascertaining a time interval between atrial and/or ventricular depolarization; a device for programming a frequency limit above which a rate of the ventricles is recognized as tachycardia; a comparison device for comparing the measured heart rate during parasympathetic stimulation to the heart rate prior to or without parasympathetic stimulation and/or to the frequency limit, which delivers an output signal when with parasympathetic stimulation the heart rate falls below the comparison value by more than a predetermined amount; and an inhibition unit which responds to the output signal to inhibit ventricular myocardial over-stimulation therapy.

U.S. Pat. No. 7,212,870 to Helland, which is incorporated herein by reference, describes an implantable lead for use with an implantable medical device, which includes a lead body with first and second electrical conductors extending between its proximal and distal ends. An electrical connector at the proximal end of the lead body includes terminals electrically connected to the first and second conductors. First and second coaxial active fixation helices are coupled to the lead body's distal end, one being an anode, the other an electrically isolated cathode. Each helix has an outer peripheral surface with alternating insulated and un-insulated portions along its length with about a half of the surface area being insulated. The un-insulated portions of the helices may be formed as a plurality of islands in the insulated portions, or as rings spaced by insulative rings, or as longitudinally extending strips spaced by longitudinally extending insulative strips.

US Patent Application Publication 2006/0206159 to Moffitt et al., which is incorporated herein by reference, describes techniques for applying neural stimulation to first and second neural stimulation sites of a heart. Nerve endings in an IVC-LA fat pad are stimulated in some embodiments using an electrode screwed into the fat pad using either an epicardial or intravascular lead, and are transvascularly stimulated in some embodiments using an intravascular electrode proximately positioned to the fat pad in a vessel such as the inferior vena cava or coronary sinus, or a lead in the left atrium. Some embodiments use an intravascularly-fed lead adapted to puncture through a vessel wall to place an electrode proximate to a target neural stimulation site.

US Patent Application Publication 2006/0217772 to Libbus et al., which is incorporated herein by reference, describes a stimulation platform, including a sensing circuit configured to sense an intrinsic cardiac signal, and a stimulation circuit configured to deliver a stimulation signal for both neural stimulation therapy and cardiac rhythm management (CRM) therapy. Neural targets in a fat pad are stimulated in some embodiments using an electrode screwed into the fat pad, and are stimulated in some embodiments using an intravenously-fed lead proximately positioned to the fat pad in a vessel such as the right pulmonary artery, right pulmonary vein, the inferior vena cava, coronary sinus, or a lead in the left atrium, for example.

US Patent Application Publication 2006/0241725 to Libbus et al., which is incorporated herein by reference, describes a presentation device such as a display screen or a printer that provides for simultaneous presentation of temporally aligned cardiac and neural signals. At least one cardiac signal in the form of a cardiac signal trace or cardiac event markers and at least one neural signal in the form of a neural signal trace or neural event markers are simultaneously presented. The cardiac signal indicates sensed cardiac electrical activities and/or cardiac stimulation pulse deliveries. The neural signal indicates sensed neural electrical activities and/or neural stimulation pulse deliveries. In one embodiment, the presentation device is part of an external system communicating with an implantable system that senses cardiac and/or neural signals and delivers cardiac and/or neural stimulation pulses.

US Patent Application Publication 2006/0271108 to Libbus et al., which is incorporated herein by reference, describes a neural stimulation system that includes a safety control system that prevents delivery of neural stimulation pulses from causing potentially harmful effects. The neural stimulation pulses are delivered to one or more nerves to control the physiological functions regulated by the one or more nerves. Examples of such harmful effects include unintended effects in physiological functions associated with autonomic neural stimulation and nerve injuries caused by excessive delivery of the neural stimulation pulses.

US Patent Application Publication 2006/0206153 to Libbus et al., which is incorporated herein by reference, describes a main lead assembly having a proximal portion adapted for connection to a device and a distal portion adapted for placement in a coronary sinus, the distal portion terminating in a distal end for placement proximal a left ventricle. Additionally, the main lead assembly includes a left ventricular electrode located at its distal end which is adapted to deliver cardiac resynchronization therapy to reduce ventricular wall stress. The main lead assembly also includes a fat pad electrode disposed along the main lead assembly a distance from the distal end to position the fat pad electrode proximal to at least one parasympathetic ganglia located in a fat pad bounded by an inferior vena cava and a left atrium. The fat pad electrode is adapted to stimulate the parasympathetic ganglia to reduce ventricular wall stress.

U.S. Pat. No. 5,334,221 to Bardy, which is incorporated herein by reference, describes a stimulator for providing stimulus pulses to the SA nodal fat pad, in response to heart rate exceeding a predetermined level, in order to reduce the ventricular rate. The device is also provided with a cardiac pacemaker to pace the ventricle in the event that the stimulus pulses reduce the heart rate below a predetermined value. The device is also provided with a feedback regulation mechanism for controlling the parameters of the stimulation pulses applied to the AV nodal fat pad, as a result of their determined effect on heart rate.

U.S. Pat. No. 7,020,530 to Ideker et al., which is incorporated herein by reference, describes a passive conductor assembly for use with an implanted device having an intra-cavitarily or trans-venously disposed electrode. The assembly can include electrical components in electrical communication therewith which provide for the manipulation, and/or modification of the electrical stimulus or waveform generated by the implanted stimulus generator, which can be designed, for example, to selectively stimulate only neural tissue, not cardiac tissue or vice versa through the same passive conductor assembly. The uninsulated portions (electrodes) of at least one conductive element are disposed in contact with the heart and/or other tissues such as neural tissue, fat pads containing post-ganglionic neural fibers, cardiac veins adjacent to neural fibers, or other electrically excitable tissues such as the stellate ganglia and the vagus. The conductive element can also run circumferentially along the atrial-ventricular groove of the heart such that the sympathetic and the parasympathetic innervation, running parallel to cardiac vasculature, can be directly stimulated or inhibited.

U.S. Pat. No. 4,161,952 to Kinney et al., which is incorporated herein by reference, describes an implantable catheter-type cardioverting electrode whose conductive discharge surface is comprised of coils of wound spring wire. An electrically conductive lead extends through the wound wire section of the electrode and has its distal end connected to the discharge coil at two locations. The proximal end of the conductive lead is adapted for connection to an implanted pulse generator.

U.S. Pat. No. 6,934,583 to Weinberg et al., which is incorporated herein by reference, describes techniques for stimulating the right vagal nerve by positioning an electrode portion of a lead proximate to the portion of the vagus nerve where the right cardiac branch is located (e.g., near or within an azygos vein, or the superior vena cava near the opening of the azygos vein) and delivering an electrical signal to an electrode portion adapted to be implanted therein. Stimulation of the right vagus nerve and/or the cardiac branch thereof act to slow the atrial heart rate. Exemplary embodiments include deploying an expandable or self-oriented electrode (e.g., a basket, an electrode umbrella, and/or an electrode spiral electrode, electrode pairs, etc).

U.S. Pat. No. 7,027,876 to Casavant et al., which is incorporated herein by reference, describes methods and endocardial screw-in leads for enabling provision of electrical stimulation to the heart, particularly the His Bundle in the intraventricular septal wall. An endocardial screw-in lead having a distal end coupled to a retractable fixation helix wherein a distal portion of the fixation helix extends beyond the lead distal end when the fixation helix is fully retracted or partially extended is positioned in proximity to the His Bundle in the septal wall. The lead body is rotated to attach the distal portion of the fixation helix into the septal wall. The fixation helix is rotated with respect to the lead body to fully extend the fixation helix so that a portion of the fixation helix is in proximity to the His Bundle, enabling provision of electrical stimulation to the His Bundle and/or to sense electrical signals of the heart traversing the His Bundle through the fixation helix.

US Patent Application Publication 2006/0241733 to Zhang et al., which is incorporated herein by reference, describes a lead that includes a lead body having an expandable section. A plurality of electrodes are disposed on the expandable section. The expandable section is adapted to expand against an inner surface of a heart so as to position at least one of the plurality of electrodes at or near an SA node of the heart.

U.S. Pat. No. 6,850,801 to Kieval et al., which is incorporated herein by reference, describes techniques for selectively and controllably reducing blood pressure, nervous system activity, and neurohormonal activity by activating baroreceptors. A baroreceptor activation device is positioned near a baroreceptor, preferably in the carotid sinus. A mapping method permits the baroreceptor activation device to be precisely located to maximize therapeutic efficacy.

An article by Lemery R et al., entitled, “Feasibility study of endocardial mapping of ganglionated plexuses during catheter ablation of atrial fibrillation,” Heart Rhythm 3:387-396 (2006), which is incorporated herein by reference, describes methods of assessing the safety and efficacy of high-frequency stimulation at mapping cardiac ganglionated plexuses in patients undergoing catheter ablation of AF. In their study, fourteen patients with a history of symptomatic AF underwent a single transseptal approach and electroanatomic mapping of the left atrium, right atrium, and coronary sinus. Using high-frequency stimulation with patients under general anesthesia (20-50 Hz, 5-15 V, pulse width 10 ms), mapping of ganglionated plexuses was performed. Radiofrequency (RF) ablation was performed during AF guided by complex fractionated atrial electrograms. Lesions were mostly delivered circumferentially in the antral area of the PVs, predominantly over and adjacent to regions of ganglionated plexuses. There was a mean of 4+/−1 (range 2-6) ganglionated plexuses per patient, and a mean total of 3+/−1 RF applications were delivered over positive vagal sites. Although a vagal response occurred infrequently during ablation (0.9%), postablation high-frequency stimulation failed to provoke a vagal response in 30 (88%) of 34 previously positive vagal sites that underwent ablation. Thus, it was concluded that ganglionated plexuses can be precisely mapped using high-frequency stimulation and are located predominantly in the path of lesions delivered during ablation of AF. Objective documentation of modification of autonomic tone can be documented in the majority of patients. Future studies were described as being required to determine the specific role of mapping and targeting of ganglionated plexuses in patients undergoing catheter ablation of AF.

The following references, all of which are incorporated herein by reference, may be of interest:
U.S. Pat. No. 4,010,755 to Preston
U.S. Pat. No. 5,170,802 to Mehra
U.S. Pat. No. 5,224,491 to Mehra
U.S. Pat. No. 5,243,980 to Mehra
U.S. Pat. No. 5,203,326 to Collins
U.S. Pat. No. 5,356,425 to Bardy et al.
U.S. Pat. No. 5,507,784 to Hill et al.
U.S. Pat. No. 6,006,134 to Hill et al.
U.S. Pat. No. 6,542,774 to Hill et al.
U.S. Pat. No. 5,690,681 to Geddes et al.
U.S. Pat. No. 5,916,239 to Geddes et al.
U.S. Pat. No. 5,700,282 to Zabara
U.S. Pat. No. 6,073,048 to Kieval et al.
U.S. Pat. No. 6,161,029 to Spreigl et al.
U.S. Pat. No. 6,317,631 to Ben-Haim et al.
U.S. Pat. No. 6,363,279 to Ben-Haim et al.
U.S. Pat. No. 6,463,324 to Ben-Haim et al.
U.S. Pat. No. 6,445,953 to Bulkes et al.
U.S. Pat. No. 7,167,748 to Ben-Haim et al.
U.S. Pat. No. 6,564,096 to Mest
U.S. Pat. No. 6,985,774 to Kieval et al.
U.S. Pat. No. 6,865,416 to Dev et al.
U.S. Pat. No. 7,087,053 to Vanney
U.S. Pat. No. 7,123,961 to Kroll et al.
U.S. Pat. No. 7,139,607 to Shelchuk
U.S. Pat. No. 7,236,821 to Cates et al.
U.S. Pat. No. 7,245,967 to Shelchuk
U.S. Pat. No. RE38,705 to Hill et al.
US Patent Application Publication 2004/0199210 to Shelchuk
US Patent Application Publication 2005/0131467 to Boveja
US Patent Application Publication 2005/0187584 to Denker et al.
US Patent Application Publication 2006/0074449 to Denker et al.
US Patent Application Publication 2005/0261672 to Deem et al.
US Patent Application Publication 2005/0273138 to To et al.
US Patent Application Publication 2006/0052831 to Fukui
US Patent Application Publication 2006/0206154 to Moffitt et al.
US Patent Application Publication 2006/0229677 to Moffitt et al.
US Patent Application Publication 2006/0247607 to Cornelius et al.
PCT Publication WO 04/52444 to Vaingast et al.
U.S. Pat. No. 6,628,987 to Hill et al.
PCT Publication WO 07/053,065 to Eckerdal et al.
US Patent Application Publication 2007/0162079 to Shemer et al.
U.S. Pat. No. 5,893,881 to Elsberry et al.
U.S. Pat. No. 5,800,470 to Stein et al.
Goldberger J J et al., “New technique for vagal nerve stimulation,” J Neurosci Methods 91(1-2):109-14 (1999)
Wallick D W et al., “Selective AV nodal vagal stimulation improves hemodynamics during acute atrial fibrillation in dogs,” Am J Physiol Heart Circ Physiol 281:H1490-H1497 (2001)
Zhang Y et al., “Optimal ventricular rate slowing during atrial fibrillation by feedback AV nodal-selective vagal stimulation,” Am J Physiol Heart Circ Physiol 282:H1102-H1110 (2002)
Zhang Y et al., “Chronic atrioventricular nodal vagal stimulation: First evidence for long-term ventricular rate control in canine atrial fibrillation model,” Circulation 112:2904-2911 (2005)
Schauerte P et al., “Ventricular rate control during atrial fibrillation by cardiac parasympathetic nerve stimulation: A transvenous approach,” Journal of the American College of Cardiology 34(7):2043-2050 (1999)
Schauerte P et al., “Catheter stimulation of cardiac parasympathetic nerves in humans: A novel approach to the cardiac autonomic nervous system,” Circulation 104:2430-2435 (2001)
Schauerte P et al., “Transvenous parasympathetic cardiac nerve stimulation for treatment of tachycardic atrial fibrillation,” Tachycarde Rhythmusstorungen 89:766-773 (2000)
Lazzara R et al., “Selective in situ parasympathetic control of the canine sinuatrial and atrioventricular node,” Circulation Research 32:393-401 (1973)
Chen S A, et al., “Intracardiac stimulation of human parasympathetic nerve fibers induces negative dromotropic effects: implication with the lesions of radiofrequency catheter ablation,” Journal of Cardiovascular Electrophysiology 9(3):245-52 (1998)
Tarver W B et al., “Clinical experience with a helical bipolar stimulating lead,” Pace, Vol. 15, October, Part 11 (1992)

### SUMMARY OF THE INVENTION

In some embodiments of the present invention, techniques are provided for applying intra-atrial parasympathetic stimulation. For some applications, the stimulation is applied to a site in an atrium of a subject, such as myocardium of the left atrium, myocardium of the right atrium, or myocardium of the interatrial septum.

In some embodiments of the present invention, a subject is identified as suffering from a cardiac condition, and intra-atrial stimulation of one or more parasympathetic epicardial fat pads is applied to treat the condition. The condition typically includes chronic heart failure (HF), atrial flutter, chronic atrial fibrillation (AF), chronic AF combined with HF, hypertension, angina, and/or an inflammatory condition of the heart. Alternatively or additionally, the techniques described herein are used post-myocardial infarct, post heart surgery, post heart transplant, during heart surgery, or during an otherwise indicated catheterization (such as PTCA). Further alternatively or additionally, the stimulation is applied to regular the production of nitric oxide (NO) (e.g., by changing the level of at least one NO synthase), such as in combination with techniques described in U.S. application Ser. No. 11/234,877, filed Sep. 22, 2005, entitled, “Selective nerve fiber stimulation,” which is assigned to the assignee of the present application and is incorporated herein by reference.

For some applications, such implantation is used for applying stimulation for preventing and/or terminating atrial fibrillation, typically by applying the stimulation to the AV node fat pad. For some applications, techniques are used that are described in U.S. patent application Ser. No. 11/657,784, filed Jan. 24, 2007 and/or U.S. patent application Ser. No. 10/560,654, filed May 1, 2006, both of which are assigned to the assignee of the present application and are incorporated herein by reference. For some applications, such epicardial implantation is used for treating a subject suffering from both heart failure and atrial fibrillation. For some applications, such stimulation is applied only when a sensed heart rate of the subject exceeds a threshold heart rate, such as about 60 BMP.

In some embodiments of the present invention, the techniques of the present invention are performed using a parasympathetic stimulation system that comprises at least one electrode assembly, which is applied to a cardiac site containing parasympathetic nervous tissue, such as an atrial site, and an implantable or external control unit. The electrode assembly comprises a lead coupled to one or more electrode contacts. For some applications, the electrode contacts are configured to be implanted in a right atrium, typically in contact with atrial muscle tissue in a vicinity of a parasympathetic epicardial fat pad. For some applications, the electrode contacts are fixed within the atrium using active fixation techniques. For some applications, the parasympathetic epicardial fat pad comprises a sinoatrial (SA) fat pad, while for other applications, the parasympathetic epicardial fat pad comprises an atrioventricular (AV) fat pad. For some applications, separate electrode assemblies, or separate electrode contacts of a single electrode assembly, are implanted in the vicinity of both the SA node fat pad and the AV node fat pad, for activating both fat pads.

In some embodiments of the present invention, the electrode assembly comprises a rotational-engagement fixation element, typically a screw-in fixation element. For some applications, the fixation element is sized such that its proximal end extends to the surface of the atrial wall when fully implanted, while for other applications, the fixation mechanism is shorter, such that its proximal end does not reach the surface of the atrial wall when fully implanted, but instead terminates inside the atrial wall. The surface of a proximal portion of the fixation element is electrically insulated, e.g., comprises a non-conductive coating, such as Teflon or silicone, around a conductive core. A distal portion of the fixation element is conductive, and serves as one of the electrode contacts.

The insulated portion of the fixation element is configured to be chronically disposed at least partially within the atrial muscle tissue, and the electrode contacts are configured to be chronically disposed in contact with the parasympathetic epicardial fat pad, typically within the fat pad. Typically, but not necessarily, the electrode contacts are positioned entirely within the fat pad, such that no portion of the electrode contacts are in contact with the atrial muscle tissue. Avoidance of direct application of current to the atrial muscle tissue generally decreases the risk of undesired cardiac capture.

In some embodiments of the present invention, an electrode contact, e.g., part of a screw-in fixation element, is configured to implanted in the atrial muscle tissue, typically either in a vicinity of the SA node fat pad 46 or the AV node fat pad. A second electrode contact is disposed on a lead which passes through the superior vena cava, such that the second electrode contact is positioned in the superior vena cava. An electric field is generated, the magnitude of which is highest in the area generally between the electrode contacts. In this manner, current generated between the electrode contacts affects the fat pad to a greater extent than the muscle tissue. Alternatively, the second electrode contact is placed in another blood vessel, such as an inferior vena cava, a coronary sinus, a right pulmonary vein, a left pulmonary vein, or a right ventricular base. Alternatively, an electrode contact positioned outside of the heart and the circulatory system in a vicinity of the fat pad (but not in physical contact with the heart or the fat pad) serves as one of the electrode contacts.

In some embodiments of the present invention, at least one electrode contact is positioned at an atrial region within an atrium (typically the right atrium, or alternatively in the left atrium) in contact with the atrial wall, within the atrial wall, and/or through the atrial wall, in a vicinity of postganglionic fibers of a parasympathetic epicardial fat pad, such as the SA node fat pad and/or the AV node fat pad, but not at or in the fat pad itself (i.e., not in contact with, or within, tissue of the cardiac wall that underlies the fat pad). Typically, but not necessarily, the atrial region is located generally between the SA node fat pad and an SA node, or generally between the AV node fat pad and an AV node. The inventors believe that stimulation of the postganglionic fibers in this region has a greater heart-rate-reduction effect than stimulation at or in the fat pads. The inventors also hypothesize that such postganglionic stimulation generally causes less afferent activation than stimulation of the fat pads or preganglionic fibers, and is thus less likely to cause side effects.

In some embodiments of the present invention, the electrode assembly comprises one or more first electrode contacts which are configured to be placed in a coronary sinus. For some applications, the first electrode contacts comprise ring electrodes, or are incorporated into one or more baskets or coronary stents. A second electrode contact (which may comprise any of the fixation elements described herein, such as a screw-in fixation element) is configured to be implanted in a vicinity of the AV node fat pad, in contact with the atrial wall, within the atrial wall, and/or through the atrial wall into the fat pad. The control unit drives a current between the second electrode contact and each of the first electrode contacts in alternation. The alternation among the first electrode contacts generally reduces the likelihood of exhausting the ganglia within the AV node fat pad.

In some embodiments of the present invention, a method for implanting an electrode contact is provided, comprising placing the electrode contact within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base. The electrode contact is advanced into a wall of the organ; a property (e.g., impedance) of tissue in a vicinity of a distal tip of the electrode contact is monitored over time. A determination is made that the tip of the electrode contact has penetrated through the wall into the fat pad upon detecting a change in the property, upon which advancement of the electrode contact is ceased.

There is therefore provided, in accordance with an embodiment of the present invention, a method including:

implanting in an atrial wall of a subject, from within an atrium, a first electrode contact in a vicinity of a parasympathetic epicardial fat pad of the subject;

implanting a second electrode contact in a body of the subject outside of a heart and a circulatory system; and

driving a current between the first and second electrode contacts, and configuring the current to cause parasympathetic activation of the fat pad.

In an embodiment, implanting the first electrode contact includes implanting, from within the atrium, a fixation element including a screw that includes the first electrode contact.

For some applications, implanting the second electrode includes implanting the second electrode at a location that is not in physical contact with the heart or the fat pad.

For some applications, configuring the current includes configuring the current such that a pulse frequency, an amplitude, and a pulse width thereof have a product that is less than 12 Hz*mA*ms, and such that the current reduces a heart rate of the subject by at least 10% compared to a baseline heart rate of the subject in the absence of the application of the current.

For some applications, implanting the second electrode contact includes implanting the second electrode contact in a vicinity of left sides of right ribs of the subject. Alternatively or additionally, implanting the second electrode contact includes implanting the second electrode contact under right ribs of the subject. Further alternatively or additionally, implanting the second electrode contact includes subcutaneously implanting the second electrode contact on a right side of a chest of the subject.

For some applications, implanting the fixation element and the second electrode contact includes implanting the fixation element and the second electrode contact such that a distance between the first and second electrode contacts is no more than 4 cm.

For some applications, driving the current includes configuring the current such that the first electrode contact serves as a cathode, and the second electrode contact as an anode.

For some applications, implanting the second electrode element includes implanting the second electrode element before implanting the fixation element, and implanting the fixation element includes: positioning the first electrode contact at a plurality of locations of in the vicinity of the fat pad; while the first electrode contact is positioned at each of the locations, driving the current between the first and second electrode contacts and sensing a vagomimetic effect; and implanting the fixation element such that the first electrode contact is positioned at the one of the locations at which a greatest vagomimetic effect was sensed.

There is further provided, in accordance with an embodiment of the present invention, apparatus including:

a first electrode contact, configured to be implanted, from within an atrium, in an atrial wall of a subject in a vicinity of a parasympathetic epicardial fat pad of the subject;

a second electrode contact, configured to be implanted in a body of the subject outside of a heart and a circulatory system; and

a control unit, configured to:

drive a current between the first and second electrode contacts, and

configure the current to cause parasympathetic activation of the fat pad.

There is still further provided, in accordance with an embodiment of the present invention, a method including:

implanting in an atrial wall of a subject, from within an atrium, a first electrode contact in a vicinity of a parasympathetic epicardial fat pad of the subject;

placing a second electrode contact within an organ of a circulatory system selected from the group consisting of: a superior vena cava, an inferior vena cava, a coronary sinus, a right pulmonary vein, a left pulmonary vein, and a right ventricular base; and

driving a current between the first and second electrode contacts, and configuring the current to cause parasympathetic activation of the fat pad.

In an embodiment, implanting the first electrode contact includes implanting, from within the atrium, a fixation element including a screw that includes the first electrode contact.

For some applications, configuring the current includes configuring the current such that a pulse frequency, an amplitude, and a pulse width thereof have a product that is less than 12 Hz*mA*ms, and such that the current reduces a heart rate of the subject by at least 10% compared to a baseline heart rate of the subject in the absence of the application of the current.

For some applications, the site includes the coronary sinus, the fat pad includes an atrioventricular (AV) node fat pad, placing includes placing the second electrode contact in the coronary sinus, and implanting includes implanting the first electrode contact in the vicinity of the AV node fat pad.

For some applications, implanting and placing include implanting the first electrode contact and placing the second electrode contact such that a distance between the first and second electrode contacts is no more than 2 cm.

For some applications, driving the current includes configuring the current such that the first electrode contact serves as a cathode, and the second electrode contact as an anode.

There is additionally provided, in accordance with an embodiment of the present invention, apparatus including:

a first electrode contact, configured to be implanted, from within an atrium, in an atrial wall of a subject in a vicinity of a parasympathetic epicardial fat pad of the subject;

a second electrode contact, configured to be placed within an organ of a circulatory system selected from the group consisting of: a superior vena cava, an inferior vena cava, a coronary sinus, a right pulmonary vein, a left pulmonary vein, and a right ventricular base; and

a control unit, configured to:

drive a current between the first and second electrode contacts, and

configure the current to cause parasympathetic activation of the fat pad.

There is yet additionally provided, in accordance with an embodiment of the present invention, a method including:

implanting in an atrial wall of a subject, from within an atrium, at least two fixation elements including respective screws that include respective electrode contacts, such that the electrode contacts are positioned in a vicinity of a parasympathetic epicardial fat pad of the subject; and

driving a current between the electrode contacts, and configuring the current to cause parasympathetic activation of the fat pad.

In an embodiment, implanting comprises implanting at least one of the fixation elements such that the electrode contact thereof is positioned entirely within the fat pad, and no other portion of the at least one of the fixation elements is in direct electrical contact with tissue of the atrial wall.

For some applications, at least one of the screws has a proximal portion having a non-conductive external surface, and a distal portion having a conductive external surface that serves as the electrode contact of the screw.

There is also provided, in accordance with an embodiment of the present invention, a method including:

placing at least three electrodes contacts at respective sites in a vicinity of a parasympathetic epicardial fat pad;

selecting a first set of at least two of the electrode contacts, and a second set of at least two of the electrode contacts, wherein the first and second sets are not identical;

during at least one stimulation period for each of 30 consecutive days, alternatingly (a) driving a current between the electrode contacts of the first set, and (b) driving the current between the electrode contacts of the second set; and

configuring the current to cause parasympathetic activation of the fat pad.

For some applications, selecting the first and second sets includes including at least one common electrode in both sets.

In an embodiment, placing includes implanting, in an atrial wall, from within an atrium, a fixation element including a screw that includes at least one of the electrode contacts, such that the at least one of the electrode contacts is positioned in the vicinity of the fat pad.

For some applications, driving the current includes configuring the current such that the at least one of the electrode contacts serves as a cathode.

For some applications, the least one of the electrode contacts includes a first one of the electrode contacts, placing includes placing second and third ones of the electrode contacts within a coronary sinus, the first set includes the first and the second ones of the electrode contacts, and the second set includes the first and the third ones of the electrode contacts.

For some applications, the least one of the electrode contacts includes a first one of the electrode contacts, and placing includes implanting second and third ones of the electrode contacts in a body of the subject outside of a heart and a circulatory system.

There is further provided, in accordance with an embodiment of the present invention, apparatus including:

an electrode assembly including an intracardiac lead and proximal and distal intracardiac electrode contacts coupled to the lead;

an intracardiac sheath sized so as to allow passage of the lead therethrough, and having a wall that includes a conducting portion through which electricity is conductible, wherein the electrode assembly and the sheath are configured such that the proximal electrode contact is aligned with the conducting portion when the distal electrode contact is advanced through the sheath at least to a distal opening of the sheath; and

a control unit, configured to drive a current between the proximal and distal electrode contacts when the proximal electrode contact is aligned with the conducting portion of the sheath.

In an embodiment of the present invention, the wall of the sheath is shaped so as to define a window that defines the conducting portion. Alternatively, the wall of the sheath includes a conductive element that serves as the conducting portion.

For some applications, the sheath is configured such that the conducing portion extends from the distal opening of the sheath for at least 1 cm in a proximal direction along the sheath. For some applications, at least a portion of the conducting portion is deflectable.

There is still further provided, in accordance with an embodiment of the present invention, a method including:

providing an electrode assembly including an intracardiac lead and proximal and distal intracardiac electrode contacts coupled to the lead;

positioning an intracardiac sheath such that at least a distal end thereof is in a heart, the sheath sized so as to allow passage of the lead therethrough, and having a wall that includes a conducting portion through which electricity is conductible;

advancing the distal electrode contact through the sheath at least to a distal opening of the sheath, such that the proximal electrode contact is aligned with the conducting portion; and

driving a current between the proximal and distal electrode contacts when the proximal electrode contact is aligned with the conducting portion of the sheath.

There is additionally provided, in accordance with an embodiment of the present invention, a method for implanting an electrode contact, including:

placing the electrode contact within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base;

advancing the electrode contact into a wall of the organ;

monitoring a property of tissue in a vicinity of a distal tip of the electrode contact over time;

making a determination that the tip of the electrode contact has penetrated through the wall into the fat pad upon detecting a change in the property; and

ceasing advancing the electrode contact responsively to the determination.

In an embodiment, the organ includes the right atrium, and placing includes implanting, within the right atrium, a fixation element including a screw that includes the electrode contact.

For some applications, the electrode contact includes a first electrode contact, and monitoring the property includes placing a second electrode contact in a vicinity of the first electrode contact, and monitoring impedance between the first and second electrode contacts.

For some applications, ceasing the advancing includes advancing the tip slightly further into the fat pad before ceasing the advancing, and monitoring the property includes continuing to monitor the property after making the determination, and further including: making a subsequent determination that the tip of the electrode contact has penetrated through the fat pad into a pericardial space beyond the fat pad upon detecting a subsequent change in the property; and withdrawing the tip of the electrode contact back into the fat pad responsively to the subsequent determination.

For some applications, ceasing advancing includes leaving the electrode contact in contact with the fat pad for at least one week.

There is yet additionally provided, in accordance with an embodiment of the present invention, apparatus including:

an electrode contact configured to penetrate, from with an atrium of a subject, an atrial wall at a penetration site;

a lead coupled to the electrode contact at a distal end of the lead, the lead including an element having a greater diameter than the electrode contact, and configured to surround the penetration site and reduce potential blood flow through the penetration site; and

a control unit configured to drive the electrode contact to apply stimulation to tissue of the subject, and configure the stimulation to cause parasympathetic activation.

For some applications, the apparatus further includes a sheath surrounding the electrode contact, and the element is connected to the sheath, and is configured to be pushed forward to press against the atrial wall after the electrode contact is implanted. For some applications, at least a portion of the electrode that penetrates the atrial wall has a proximal portion having a greater cross-sectional area than a distal portion thereof.

There is also provided, in accordance with an embodiment of the present invention, apparatus including:

a helically-shaped intracardiac screw-in fixation element, including, at a distal tip thereof, a bipolar electrode, which includes:
an outer electrode contact; and
and an inner electrode contact arranged coaxially within the outer electrode contact, and electrically isolated from the outer electrode contact; and

a control unit, configured to drive the bipolar electrode to apply cardiac stimulation.

There is further provided, in accordance with an embodiment of the present invention, a method including:

providing an electrode assembly including a lead and at least two electrode contacts coupled to the lead;

positioning the electrode assembly such that the at least two electrode contacts are within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

advancing the at least two electrode contacts into a wall of the organ.

There is still further provided, in accordance with an embodiment of the present invention, a method including:

coupling, from within an atrium of a subject, a distal portion of at least one electrode assembly to an atrial wall, such that at least one electrode contact of the electrode assembly is in contact with tissue in a vicinity of a parasympathetic epicardial fat pad;

driving the at least one electrode contact to apply electrical stimulation to the tissue; and

configuring the stimulation such that a pulse frequency, an amplitude, and a pulse width thereof have a product that is less than 12 Hz*mA*ms, and such that the stimulation reduces a heart rate of the subject by at least 10% compared to a baseline heart rate of the subject in the absence of the stimulation.

For some applications, configuring includes configuring the stimulation to reduce the heart rate by at least 20% compared to the baseline heart rate.

There is additionally provided, in accordance with an embodiment of the present invention, a method including:

chronically implanting at least one screw electrode within an atrium of a subject;

driving the at least one electrode to apply stimulation to tissue of the atrium;

configuring the stimulation to stimulate at least one vagal ganglion plexus (GP) of the subject; and

setting a duration of the stimulation to be between 1 and 10 microseconds.

There is yet additionally provided, in accordance with an embodiment of the present invention, a method including:

implanting, from within a right atrium, an electrode contact in an atrial wall at a site that is in a vicinity of postganglionic fibers of a parasympathetic epicardial fat pad, and that does not underlie the fat pad;

driving the electrode contact to apply a current to the postganglionic fibers; and

configuring the current to activate the postganglionic fibers.

For some applications, the fat pad includes a sinoatrial (SA) node fat pad, and the site is generally between the SA node fat pad and an SA node. For some applications, the site is at least 1 mm from the SA node.

In an embodiment, the fat pad includes a atrioventricular (AV) node fat pad, and the site is generally between the AV node fat pad and an AV node. For some applications, the site is at least 1 mm from the AV node.

For some applications, the site is at least 1 mm from a region of an interior surface of the atrial wall that underlies the fat pad.

There is also provided, in accordance with an embodiment of the present invention, a method including:

implanting in an atrial wall of a subject, from within an atrium, at least two fixation elements including respective screws that include respective electrode contacts, such that the electrode contacts are positioned in vicinities of respective vagomimetic sites; and

driving the electrode contacts to apply electrical stimulation to the respective vagomimetic sites, and configuring the stimulation to cause parasympathetic activation of the sites.

For some applications, the at least two fixation elements include at least three fixation elements, and implanting includes implanting the at least three fixation elements in the atrial wall, such that the respective electrode contacts are positioned in the vicinities of the respective vagomimetic sites.

For some applications, driving includes simultaneously driving all of the electrode contacts to apply the stimulation to the respective sites. Alternatively, driving includes driving each of the electrode contacts to apply the stimulation to its respective site during a local refractory period at the site.

For some applications, implanting includes implanting the fixation elements such that a first one of the electrode contacts is positioned in a vicinity of an SA node fat pad, and a second one of the electrode contacts is positioned in a vicinity of the AV node fat pad. For some applications, implanting includes identifying that the subject suffers from heart failure and paroxysmal atrial fibrillation (AF), and implanting responsively to the identifying. For some applications, driving includes: detecting whether the subject is currently in normal sinus rhythm (NSR) or experiencing an episode of the AF; upon detecting that the subject is experiencing the episode of the AF, driving the second one of the electrode contacts to apply the stimulation to the AV node fat pad; and upon detecting that the subject is currently in NSR, driving the first one of the electrode contacts to apply the stimulation to the SA node fat pad.

For some applications, configuring the stimulation includes: sensing a measure of cardiac performance of the subject; and responsively to the measure, configuring one or more parameters of the stimulation applied by the second one of the electrode contacts to the AV node fat pad to cause an improvement in the sensed measure of cardiac performance.

There is further provided, in accordance with an embodiment of the present invention, apparatus including:

first and second electrode contacts, configured to be implanted, from within a right atrium of a subject, in an atrial wall at respective sites that are in respective vicinities of an sinoatrial (SA) node fat pad and an atrioventricular (AV) node fat pad; and

a control unit, configured to:

detect an episode of non-sinus atrial tachycardia, and

responsively to the detection, restore normal sinus rhythm (NSR) of the subject by:

driving the first and second electrode contacts to apply respective parasympathetic stimulation signals to the sites, and

configuring the parasympathetic stimulation signals to activate parasympathetic nervous tissue of the SA node and AV node fat pads sufficiently to restore the NSR.

For some applications, the non-sinus atrial tachycardia includes atrial fibrillation (AF), and the control unit is configured to detect the episode of the AF. Alternatively or additionally, the non-sinus atrial tachycardia includes atrial flutter, and the control unit is configured to detect the episode of the atrial flutter.

There is still further provided, in accordance with an embodiment of the present invention, a method including:

implanting, from within a right atrium, first and second electrode contacts in an atrial wall of a subject at respective sites that are in respective vicinities of an sinoatrial (SA) node fat pad and an atrioventricular (AV) node fat pad; and

detecting an episode of non-sinus atrial tachycardia of the subject; and

responsively to the detection, restoring normal sinus rhythm (NSR) of the subject by:

driving the first and second electrode contacts to apply respective parasympathetic stimulation signals to the sites, and

configuring the parasympathetic stimulation signals to activate parasympathetic nervous tissue of the SA node and AV node fat pads sufficiently to restore the NSR.

There is additionally provided, in accordance with an embodiment of the present invention, apparatus including:

an electrode contact, configured to be implanted, from within a right atrium of a subject, in an atrial wall at a site that is in a vicinity of an atrioventricular (AV) node fat pad; and

a control unit, configured to:

detect whether the subject is experiencing atrial fibrillation or is in normal sinus rhythm (NSR),

responsively to detecting that the subject is experiencing AF, drive the electrode contact to applying stimulation to the site, and configure the stimulation to cause parasympathetic activation of the fat pad at a strength sufficient to reduce a heart rate of the subject; and

responsively to detecting that the subject is in NSR, converting the subject to AF by driving the electrode contact to apply a pacing signal to the site having a frequency of at least 1.5 Hz.

There is yet additionally provided, in accordance with an embodiment of the present invention, a method including:

implanting, from within a right atrium of a subject, an electrode contact in an atrial wall at a site that is in a vicinity of an atrioventricular (AV) node fat pad;

detecting whether the subject is experiencing atrial fibrillation or is in normal sinus rhythm (NSR);

responsively to detecting that the subject is experiencing AF, driving the electrode contact to applying stimulation to the site, and configuring the stimulation to cause parasympathetic activation of the fat pad at a strength sufficient to reduce a heart rate of the subject; and

responsively to detecting that the subject is in NSR, converting the subject to AF by driving the electrode contact to apply a pacing signal to the site having a frequency of at least 1.5 Hz.

There is also provided, in accordance with an embodiment of the present invention, a method including:

identifying that a subject suffers from at least one condition selected from the group consisting of: heart failure (HF) combined with atrial fibrillation, HF combined with atrial flutter, hypertension, and an inflammatory condition of the heart;

responsively to the identifying:

implanting in an atrial wall of the subject, from within an atrium, a fixation element including a screw that includes at least one electrode contact, such that the at least one electrode contact is positioned in a vicinity of a parasympathetic epicardial fat pad; and

treating the condition by driving the at least one electrode to apply electrical stimulation to the fat pad.

There is further provided, in accordance with an embodiment of the present invention, a method including:

identifying a clinical benefit for a subject of an increased eNOS level;

responsively to the identifying:

implanting in an atrial wall of the subject, from within an atrium, a fixation element including a screw that includes at least one electrode contact, such that the at least one electrode contact is positioned in a vicinity of a parasympathetic epicardial fat pad; and

increasing the eNOS level by driving the at least one electrode to apply electrical stimulation to the fat pad.

There is still further provided, in accordance with an embodiment of the present invention, a method including:

identifying a clinical benefit for a subject of a reduced iNOS level and a reduced nNOS level in cardiac tissue;

responsively to the identifying:

implanting in an atrial wall of the subject, from within an atrium, a fixation element including a screw that includes at least one electrode contact, such that the at least one electrode contact is positioned in a vicinity of a parasympathetic epicardial fat pad; and

reducing the iNOS and nNOS levels by driving the at least one electrode to apply electrical stimulation to the fat pad.

There is additionally provided, in accordance with an embodiment of the present invention, a method including:

implanting in an atrial wall of a subject, from within an atrium, at least one electrode contact at site in a vicinity of a parasympathetic epicardial fat pad;

driving the electrode contact to apply an electrical signal to the site; and

configuring the signal to both pace the heart and cause parasympathetic activation of the fat pad.

For some applications, configuring includes configuring the signal to include bursts, each of which includes a plurality of pulses, and configuring one or more initial pulses of each of the bursts to pace the heart.

There is yet additionally provided, apparatus including:

an intravascular lead;

a first electrode contact coupled to the lead at a distal end thereof, and configured to be positioned in a right atrium in a vicinity of a parasympathetic epicardial fat pad selected from the group consisting of: an atrioventricular (AV) node fat pad, and a sinoatrial (SA) node fat pad;

a second electrode contact coupled to the lead within 2 cm of the first lead;

a third electrode contact coupled to the lead such that the second electrode contact is between the first and third electrode contacts, the third electrode contact configured to be positioned within an organ selected from the group consisting of: a superior vena cava, and a right atrium in a vicinity of the superior vena cava; and

a control unit, configured to:

sense a commencement of a P-wave using the third electrode contact and at least one electrode contact selected from the group consisting of: the first electrode contact, and the second electrode contact, and

responsively to the sensing of the commencement of the P-wave, drive a current between the first and second electrode contacts, and configure the current to cause parasympathetic activation of the fat pad.

For some applications, the control unit is configured to drive the current within 30 ms of the sensing of the commencement of the P-wave.

There is also provided, in accordance with an embodiment of the present invention, a method including:

positioning a first electrode contact coupled to an intravascular lead at a distal of the lead in a right atrium in a vicinity of a parasympathetic epicardial fat pad selected from the group consisting of: an atrioventricular (AV) node fat pad, and a sinoatrial (SA) node fat pad, wherein a second electrode contact is coupled to the lead within 2 cm of the first lead;

positioning within an organ a third electrode contact coupled to the lead such that the second electrode contact is between the first and third electrode contacts, the organ selected from the group consisting of: a superior vena cava, and a right atrium in a vicinity of the superior vena cava;

sensing a commencement of a P-wave using the third electrode contact and at least one electrode contact selected from the group consisting of: the first electrode contact, and the second electrode contact; and

responsively to the sensing of the commencement of the P-wave, driving a current between the first and second electrode contacts, and configure the current to cause parasympathetic activation of the fat pad.

There is further provided, in accordance with an embodiment of the present invention, a method for implanting an electrode assembly having at least one electrode contact, including:

positioning the electrode assembly such that the at least one electrode contact is within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

advancing the electrode contact into a wall of the organ until the electrode contact is positioned entirely within the fat pad, and no other portion of the electrode assembly is in direct electrical contact with tissue of the wall.

For some applications, the at least one electrode contact includes at least two electrode contacts, and the electrode assembly includes a fixation element including a screw that includes the at least two electrode contacts.

For some applications, the electrode assembly includes a fixation element including a screw having a proximal portion having a non-conductive external surface, and a distal portion having a conductive external surface that serves as the at least one electrode contact.

There is still further provided, in accordance with an embodiment of the present invention, a method including:

implanting at least one electrode contact within an atrium of a subject in a vicinity of an interatrial groove; and

during at least one stimulation period per day over a thirty-day period, driving the electrode contact to apply stimulation to tissue of the subject, and configuring the stimulation to cause parasympathetic activation.

For some applications, chronically implanting the at least one electrode contact in the vicinity includes chronically implanting the at least one electrode contact within 2 mm of the groove. For some applications, chronically implanting the at least one electrode contact in the vicinity includes chronically implanting the at least one electrode contact in physical contact with the groove.

There is additionally provided, in accordance with an embodiment of the present invention, apparatus including:

one or more electrode contacts, configured to be placed within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

a control unit, configured to:

drive the electrode contacts to apply to the fat pad a burst of pulses including one or more initial pulses followed by one or more subsequent pulses,

set a preconditioning strength of the initial pulses to be insufficient to cause parasympathetic activation of the fat pad, and

set an activating strength of the subsequent pulses to be sufficient to cause the parasympathetic activation.

There is yet additionally provided, in accordance with an embodiment of the present invention, a method including:

placing one or more electrode contacts within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base;

driving the electrode contacts to apply to the fat pad a burst of pulses including one or more initial pulses followed by one or more subsequent pulses;

setting a preconditioning strength of the initial pulses to be insufficient to cause parasympathetic activation of the fat pad; and

setting an activating strength of the subsequent pulses to be sufficient to cause the parasympathetic activation.

For some applications, setting the strength of the initial and subsequent pulses includes: during a calibration procedure, driving the electrode contacts to apply a plurality of calibration bursts at respective calibration strengths; sensing whether the calibration bursts cause a vagomimetic effect; finding a minimal strength necessary to cause the vagomimetic effect; and setting the preconditioning strength to be less than the minimal strength, and the activating strength to be at least the minimal strength.

There is also provided, in accordance with an embodiment of the present invention, apparatus including:

one or more electrode contacts, configured to be placed within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

a control unit, configured to:

drive the electrode contacts to apply a plurality of bursts to the fat pad,

set a frequency of the bursts to be less than or equal to 2.5 Hz, and

configure each of the bursts to include between 2 and 20 pulses, and to have a pulse repetition interval (PRI) of between 1 ms and 30 ms.

For some applications, the control unit is configured to set the burst frequency to be less than or equal to 2 Hz.

There is further provided, in accordance with an embodiment of the present invention, a method including:

placing one or more electrode contacts within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base;

driving the electrode contacts to apply a plurality of bursts to the fat pad;

setting a frequency of the bursts to be less than or equal to 2.5 Hz; and

configuring each of the bursts to include between 2 and 20 pulses, and to have a pulse repetition interval (PRI) of between 1 ms and 30 ms.

There is still further provided, in accordance with an embodiment of the present invention, apparatus including:

at least one electrode contact, configured to be implanted, from within a right atrium of a subject, in an atrial wall in a vicinity of a parasympathetic epicardial fat pad; and

a control unit, configured to:

drive the at least one electrode contact to apply bursts of pulses without synchronizing the bursts with any feature of a cardiac cycle of the subject,

configure a burst frequency to be no more than 2.5 Hz, and

configure a pulse repetition interval (PRI) to be no more than 30 ms.

There is additionally provided, in accordance with an embodiment of the present invention, a method including:

implanting, from within a right atrium of a subject, at least one electrode contact in an atrial wall in a vicinity of a parasympathetic epicardial fat pad;

driving the at least one electrode contact to apply bursts of pulses without synchronizing the bursts with any feature of a cardiac cycle of the subject;

configuring a burst frequency to be no more than 2.5 Hz; and

configure a pulse repetition interval (PRI) to be no more than 30 ms.

There is yet additionally provided, in accordance with an embodiment of the present invention, apparatus including:

an intracardiac screw-in electrode assembly including:
an outer helical fixation element having a first radius, and including a first intracardiac electrode contact; and
an inner helical fixation element having a second radius less than the first radius, and including a second intracardiac electrode contact,
wherein the inner helical fixation element is positioned within the outer helical fixation element; and

a control unit, configured to drive a current between the first and second electrode contacts, and to configure the current to provide cardiac stimulation.

For some applications, the electrode assembly is configured such that the outer and inner helical fixation members are independently rotatable.

There is also provided, in accordance with an embodiment of the present invention, a method including:

chronically implanting at least one electrode contact within an atrium of a subject in a vicinity of a parasympathetic epicardial fat pad;

driving the at least one electrode contact to apply stimulation to tissue of the atrium;

determining whether the stimulation activates a phrenic nerve of the subject; and

responsively to finding that the stimulation activates the phrenic nerve, configuring at least one parameter of the stimulation so as to not activate the phrenic nerve.

For some applications, driving includes configuring the stimulation to cause parasympathetic activation.

There is further provided, in accordance with an embodiment of the present invention, a method including:

implanting in an atrial wall of a subject, from within an atrium, at least one electrode contact in a vicinity of a parasympathetic epicardial fat pad of the subject;

sensing, using the at least one electrode, an electrogram, and analyzing the electrogram;

upon finding that the electrogram is characteristic of atrial electrical activity, driving the at least one electrode contact to apply stimulation, and configuring the stimulation to cause parasympathetic activation of the fat pad; and

upon finding that the electrogram is not characteristic of the atrial electrical activity, withholding driving the at least one electrode contact to apply the stimulation.

For some applications, sensing includes withhold applying the stimulation during a sensing period having a duration of at least 2 seconds, and sensing during the sensing period.

There is still further provided, in accordance with an embodiment of the present invention, apparatus including:

at least one electrode contact, configured to be implanted, from within an atrium, in an atrial wall of a subject in a vicinity of a parasympathetic epicardial fat pad of the subject; and

a control unit, configured to:

sense, using the at least one electrode, an electrogram, and analyze the electrogram,

upon finding that the electrogram is characteristic of atrial electrical activity, drive the at least one electrode contact to apply stimulation, and configuring the stimulation to cause parasympathetic activation of the fat pad, and

upon finding that the electrogram is not characteristic of the atrial electrical activity, withhold driving the at least one electrode contact to apply the stimulation.

There is additionally provided, in accordance with an embodiment of the present invention, a method including:

positioning two electrode contacts within an atrium of a subject at respective locations against a wall of the atrium in a vicinity of a parasympathetic epicardial fat pad;

performing a plurality of times the steps of:
(a) separately driving the two electrode contacts to apply stimulation to the wall, and determining respective heart-rate-lowering effects of the stimulation applied by the two electrode contacts;
(b) repositioning at least one other location against the wall whichever of the electrodes achieved a lesser heart-rate-lowering effect, while leaving the other of the electrode contacts at its location against the wall;

chronically implanting one of the electrode contacts at its location, and removing the other of the electrodes from the atrium.

For some applications, implanting includes again performing step (a), and implanting whichever of the electrode contacts achieved a greater heart-rate-lowering effect, at the location of the electrode.

For some applications, implanting includes identifying that the respective heart-rate-lowering effects have converged. Alternatively, implanting includes, upon finding that the respective heart-rate-lowering effects have not converged, implanting whichever of the electrode contacts achieved a greater heart-rate-lowering effect, at the location of the electrode, and removing the other of the electrodes from the atrium.

For some applications, performing the steps the plurality of times includes repositioning each of the electrode contacts at least once.

In an embodiment, positioning the electrode contacts includes placing at least one of the electrode contacts in a sheath that includes at least one conducting portion through which electricity is conductible, and driving the electrode contacts to apply the stimulation includes driving the at least one electrode contact to apply the stimulation through the at least one conducting portion. For some applications, the sheath is shaped so as to define at least one window that defines the at least one conducting portion. For some applications, the sheath includes a conductive element that serves as the at least one conducting portion.

The present invention will be more fully understood from the following detailed description of embodiments thereof, taken together with the drawings, in which:

### BRIEF DESCRIPTION OF THE DRAWINGS

FIG. 1 is a schematic illustration of a parasympathetic stimulation system for stimulating autonomic nervous tissue from at least partially within a heart, in accordance with an embodiment of the present invention;

FIGS. 2A-C are schematic illustrations of configurations of an electrode assembly of the system of FIG. 1, in accordance with respective embodiments of the present invention;

FIGS. 3A-C are schematic illustrations of a screw-in fixation elements of the system of FIG. 1, in accordance with respective embodiments of the present invention;

FIGS. 4A-B are schematic illustrations of electrode assemblies configured to minimize the risk of bleeding, in accordance with respective embodiments of the present invention;

FIG. 5A is a schematic illustration of another parasympathetic stimulation system, in accordance with an embodiment of the present invention;

FIG. 5B is a schematic illustration of an alternative configuration of the system of FIG. 5A, in accordance with another embodiment of the present invention;

FIG. 6 is a schematic illustration of yet another parasympathetic stimulation system, in accordance with an embodiment of the present invention;

FIG. 7 is a schematic illustration of a sheath, in accordance with an embodiment of the present invention;

FIG. 8 is a schematic illustration of a parasympathetic stimulation system for stimulation of postganglionic fibers, in accordance with an embodiment of the present invention;

FIG. 9 is a schematic illustration of tripolar ganglion plexus (GP) electrode assembly, in accordance with an embodiment of the present invention;

FIG. 10 is a schematic illustration of an atrial region for stimulation of postganglionic fibers, in accordance with an embodiment of the present invention;

FIG. 11 is a schematic illustration of yet another configuration of the stimulation system of FIG. 1, in accordance with an embodiment of the present invention;

FIG. 12 is a flow chart schematically illustrating a method for implanting an electrode assembly at a desired position in an atrial fat pad, in accordance with an embodiment of the present invention; and

FIGS. 13A-G are graphs illustrating electrical data recorded and/or analyzed in accordance with respective embodiments of the present invention.

### DETAILED DESCRIPTION OF EMBODIMENTS

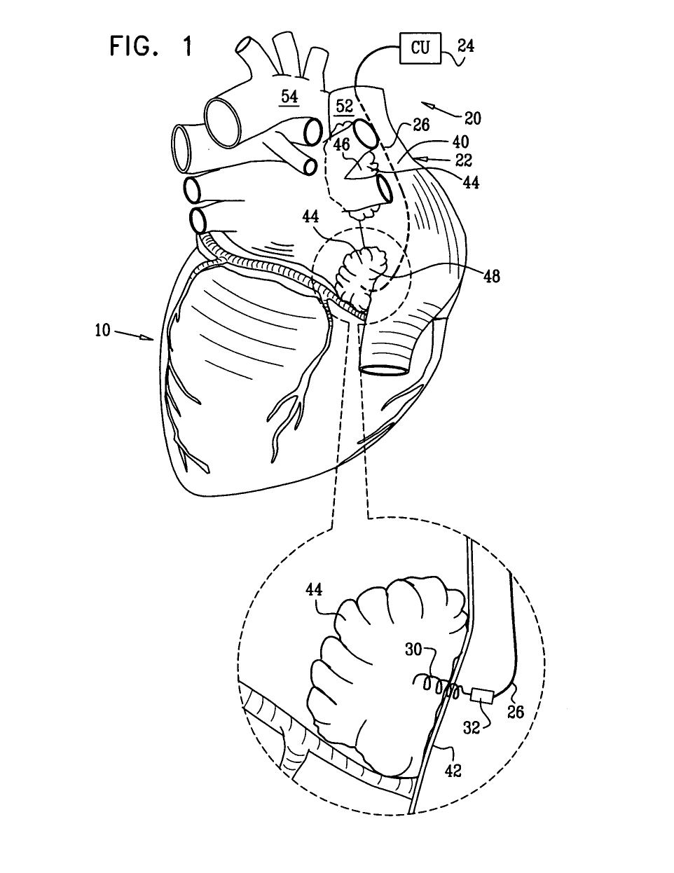

FIG. 1 is a schematic illustration of a parasympathetic stimulation system 20 for stimulating autonomic nervous tissue from at least partially within a heart 10, in accordance with an embodiment of the present invention. System 20 comprises at least one electrode assembly 22, which is applied to a cardiac site containing parasympathetic nervous tissue, such as an atrial site, and an implantable or external control unit 24. Electrode assembly 22 comprises a lead 26 coupled to one or more electrode contacts 30 and 32. Lead 26 is typically introduced into the heart using an introducer, such as a catheter or sheath.

In an embodiment of the present invention, electrode contacts 30 and 32 are configured to be implanted in a right atrium 40, typically in contact with atrial muscle tissue 42 in a vicinity of a parasympathetic epicardial fat pad 44. For some applications, electrode contacts 30 and 32 are fixed within atrium 40 using active fixation techniques. For some applications, parasympathetic epicardial fat pad 44 comprises a sinoatrial (SA) fat pad 46, while for other applications, parasympathetic epicardial fat pad 44 comprises an atrioventricular (AV) fat pad 48. For still other applications, the parasympathetic epicardial fat pad comprises an SVC-AO fat pad located in a vicinity of a junction between a superior vena cava 52 and an aorta 54 (SVC-AO fat pad not shown in figure). Alternatively, separate electrode assemblies 22, or separate electrode contacts of a single electrode assembly 22, are implanted in the vicinity of both SA node fat pad 46 and AV node fat pad 48, for activating both fat pads, such as described hereinbelow.

In an embodiment of the present invention, control unit 24 applies the parasympathetic stimulation responsively to one or more sensed parameters. Control unit 24 is typically configured to commence or halt stimulation, or to vary the amount and/or timing of stimulation in order to achieve a desired target heart rate, typically responsively to configuration values and on parameters including one or more of the following:
Heart rate—the control unit can be configured to drive electrode assembly 22 to stimulate the fat pad(s) only when the heart rate exceeds a certain value.
ECG readings—the control unit can be configured to drive electrode assembly 22 to stimulate the fat pad(s) responsively to certain ECG readings, such as readings indicative of designated forms of arrhythmia. Additionally, ECG readings are typically used for achieving a desire heart rate.
Blood pressure—the control unit can be configured to regulate the current applied by electrode assembly 22 to the fat pad(s) when blood pressure exceeds a certain threshold or falls below a certain threshold.
Indicators of decreased cardiac contractility—these indicators include left ventricular pressure (LVP). When LVP and/or d(LVP)/dt exceeds a certain threshold or falls below a certain threshold, control unit 24 can drive electrode assembly 22 to regulate the current applied by electrode assembly 22 to the fat pad(s).
Motion of the subject—the control unit can be configured to interpret motion of the subject as an indicator of increased exertion by the subject, and appropriately reduce parasympathetic stimulation of the heart in order to allow the heart to naturally increase its rate.
Heart rate variability—the control unit can be configured to drive electrode assembly 22 to stimulate the fat pad(s) responsively to heart rate variability, which is typically calculated responsively to certain ECG readings.
Norepinephrine concentration—the control unit can be configured to drive electrode assembly 22 to stimulate the fat pad(s) responsively to norepinephrine concentration.
Cardiac output—the control unit can be configured to drive electrode assembly 22 to stimulate the fat pad(s) responsively to cardiac output, which is typically determined using impedance cardiography.
Baroreflex sensitivity—the control unit can be configured to drive electrode assembly 22 to stimulate the fat pad(s) responsively to baroreflex sensitivity.
LVEDP—the control unit can be configured to drive electrode assembly 22 to stimulate the fat pad(s) responsively to LVEDP, which is typically determined using a pressure gauge.

The parameters and behaviors included in this list are for illustrative purposes only, and other possible parameters and/or behaviors will readily present themselves to those skilled in the art, who have read the disclosure of the present patent application.

In an embodiment of the present invention, control unit 24 is configured to drive electrode assembly 22 to stimulate the fat pad(s) so as to reduce the heart rate of the subject towards a target heart rate. The target heart rate is typically (a) programmable or configurable, (b) determined responsive to one or more sensed physiological values, such as those described hereinabove (e.g., motion, blood pressure, etc.), and/or (c) determined responsive to a time of day or circadian cycle of the subject. Parameters of stimulation are varied in real time in order to vary the heart-rate-lowering effects of the stimulation. For example, such parameters may include the amplitude of the applied current. Alternatively or additionally, in an embodiment of the present invention, the stimulation is applied in bursts (i.e., series of pulses), which are synchronized or are not synchronized with the cardiac cycle of the subject.

In some embodiments of the present invention, the control unit senses an ECG and/or a local cardiac electrogram, and applies the vagomimetic stimulation during the atrial effective refractory period (AERP) or local refractory period. The stimulation thus causes a vagomimetic effect without causing cardiac capture.

Typical parameters include the following:
Timing of the stimulation within the cardiac cycle. Delivery of each of the bursts typically begins after a fixed or variable delay following an ECG feature, such as each R- or P-wave. For some applications, the delay is between about 0 and about 100 ms after the P-wave.
Pulse duration (width). Longer pulse durations typically result in a greater heart-rate-lowering effect. For some applications, the pulse duration is between about 0.001 ms and about 5 ms, such as between about 0.1 ms and about 2 ms, e.g., about 0.5 ms.
Pulse repetition interval within each burst. Maintaining a pulse repetition interval (the time from the initiation of a pulse to the initiation of the following pulse within the same burst) greater than about 1 ms generally results in better stimulation effectiveness for multiple pulses within a burst. For some applications, the pulse repetition interval is between about 1 and about 20 ms, such as between about 3 and about 10 ms, e.g., about 6 ms.
Pulses per trigger (PPT). A greater PPT (the number of pulses in each burst after a trigger such as an P-wave) typically results in a greater heart-rate-lowering effect. For some applications, PPT is between about 1 and about 20 pulses, such as between about 2 and about 10 pulses, e.g., 5 pulses. For some applications, PPT is varied while pulse repetition interval is kept constant.
Amplitude. A greater amplitude of the signal applied typically results in a greater heart-rate-lowering effect. The amplitude is typically between about 0.1 and about 10 milliamps, e.g., between about 1 and about 3 milliamps, such as about 2 milliamps.
Duty cycle (number of bursts per heart beat). Application of stimulation every heartbeat (i.e., with a duty cycle of 1) typically results in a greater heart-rate-lowering effect. For less heart rate reduction, stimulation is applied less frequently than every heartbeat (e.g., duty cycle=60%-90%), or only once every several heartbeats (e.g., duty cycle=5%-40%).
“On”/“off” ratio and timing. For some applications, the device operates intermittently, alternating between “on” and “off” states, the length of each state typically being between 0 and about 1 day, such as between 0 and about 300 seconds (with a O-length “off” state equivalent to always “on”). No stimulation is applied during the “off” state. Greater heart rate reduction is typically achieved if the device is “on” a greater portion of the time.

In an embodiment of the present invention, control unit 24 set the frequency (pulses per second), amplitude, and pulse width of the signal such that the product thereof is less than 12 Hz*mA*ms, e.g., less than 6 Hz*mA*ms. Application of such a signal typically reduces the heart rate by at least 10%, e.g., at least 20%, compared to a baseline heart rate of the subject in the absence of the stimulation. It is noted that the frequency is measured in pulses per second, even for applications in which the pulses are applied in bursts, such that the pulses are not evenly distributed throughout any given second.

For some applications, values of one or more of the parameters are determined in real time, using feedback, i.e., responsive to one or more inputs, such as sensed physiological values. For example, the intermittency (“on”/“off”) parameter may be determined in real time using such feedback. The inputs used for such feedback typically include one or more of the following: (a) motion or activity of the subject (e.g., detected using an accelerometer), (b) the average heart rate of the subject, (c) the average heart rate of the subject when the device is in “off” mode, (d) the average heart rate of the subject when the device is in “on” mode, and/or (e) the time of day. The average heart rate is typically calculated over a period of at least about 10 seconds. For some applications, the average heart rate during an “on” or “off” period is calculated over the entire “on” or “off” period. For example, the device may operate in continuous “on” mode when the subject is exercising and therefore has a high heart rate, and the device may alternate between “on” and “off” when the subject is at rest. As a result, the heart-rate-lowering effect is concentrated during periods of high heart rate, and the nerve is allowed to rest when the heart rate is generally naturally lower. For some applications, the device determines the ratio of “on” to “off” durations, the duration of the “on” periods, and/or the durations of the “off” periods using feedback. Optionally, the device determines the “on”/“off” parameter in real time using the integral feedback techniques described in U.S. application Ser. No. 11/064,446, filed Feb. 22, 2005, which is assigned to the assignee of the present application and is incorporated herein by reference, mutatis mutandis.

For some applications, heart rate regulation is achieved by setting two or more parameters in combination. For example, if it is desired to apply 5.2 pulses of stimulation, the control unit may apply 5 pulses of 1 ms duration each, followed by a single pulse of 0.2 ms duration. For other applications, the control unit switches between two values of PPT, so that the desired PPT is achieved by averaging the applied PPTs. For example, a sequence of PPTs may be 5, 5, 5, 5, 6, 5, 5, 5, 5, 6, . . . , in order to achieve an effective PPT of 5.2.

In an embodiment of the present invention, control unit 24 is configured to apply the parasympathetic stimulation using feedback, as described hereinabove, wherein a parameter of the feedback is a target heart rate that is a function of an average heart rate of the subject. For some applications, the target heart rate is set equal or approximately equal to the average heart rate of the subject. Alternatively, the target heart rate is set at a rate greater than the average heart rate of the subject, such as a number of beats per minute (BPM) greater than the average heart rate, or a percentage greater than the average heart rate, e.g., about 1% to about 50% greater. Further alternatively, the target heart rate is set at a rate less than the average heart rate of the subject, such as a number of BPM less than the average heart rate, or a percentage less than the average heart rate, e.g., about 1% to about 20% less. For some applications, the target heart rate is set responsively to the duty cycle and the heart rate response of the subject. In an embodiment, control unit 24 determines the target heart rate in real time, periodically or substantially continuously, by sensing the heart rate of the subject and calculating the average heart rate of the subject. The average heart rate is typically calculated substantially continuously, or periodically. Typically, standard techniques are used for calculating the average, such as moving averages or IIR filters. The number of beats that are averaged typically varies between several beats to all beats during the past week.

For some applications, the techniques described herein are performed in combination with techniques described in above-mentioned U.S. application Ser. No. 11/064,446. In particular, control unit 24 may use feedback and parameter-setting techniques described therein.

In some embodiments of the present invention, control unit 24 is configured to test for cardiac capture, and to modify one or more stimulation parameters so as to reduce the probability of cardiac capture. In these embodiments, the stimulation is typically intended to cause parasympathetic stimulation, and causing cardiac capture (i.e., pacing) is thus undesired. For some applications in which the control unit applies one burst per cardiac cycle, the control unit tests for cardiac capture by sensing the ECG signal after completion of each burst within a cardiac cycle, either immediately after completion or after a short blanking period (e.g., less than about 60 ms, such as less than about 30 ms). If the control unit detects additional atrial depolarization waves, the control unit determines that unwanted capture has occurred, and modulates the stimulation accordingly. Typically, to modulate the stimulation, the control unit first withholds stimulation for at least one cardiac cycle (e.g., for one, two, or three cardiac cycles), and then performs stimulation again after re-detecting a P wave. If the control finds that such withholding does not prevent unwanted capture over time, the control unit changes one or more stimulation parameters, typically staging the changes until the disappearance of the capture. For some applications, the control unit first shortens the stimulation duration, such as reduces the burst duration (e.g., from about 80 ms to about 60 ms, and then even lower), until the desired disappearance of capture is achieved. If reduction of the burst duration is insufficient, the control unit then reduces the stimulation current, such as from about 10 mA to about 8 mA, and then even lower, until the desired disappearance of capture is achieved. If neither of these reductions is sufficient, the control unit may reduce the pulse width, such as from about 1 ms to about 0.3 ms.

In some embodiments of the present invention, control unit 24 senses the cardiac electrogram using a monopolar or bipolar electrode configuration. For some applications, the sensing is bipolar, and the electrode is positioned so as to sense atrial depolarization more strongly than ventricular depolarization.

In an embodiment of the present invention, control unit 24 is configured to apply respective bursts of pulses in a plurality of cardiac cycles, and to set a strength of the stimulation to be sufficient to generate a vagomimetic response, but insufficient to cause cardiac contraction (and typically insufficient to generate propagating action potentials in the myocardium of the subject). The control unit configures one or more parameters of the stimulation to set a strength thereof (e.g., current, number of pulses per burst, and/or pulse width). For some applications, the bursts are synchronized with a feature of the cardiac cycle, while for other applications, the bursts are not synchronized with a feature of the cardiac cycle.

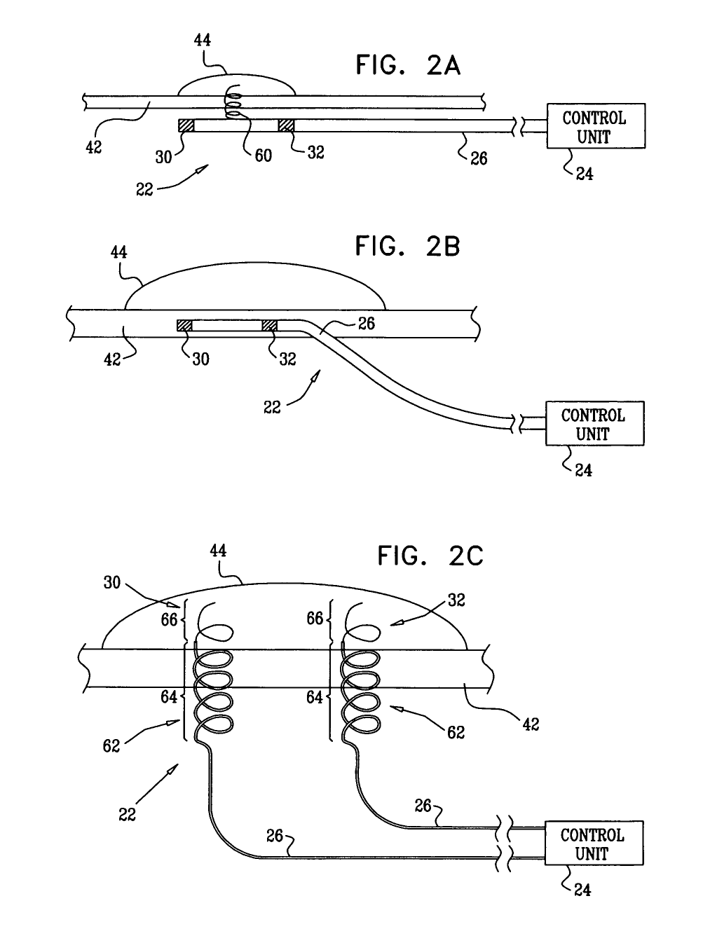

FIGS. 2A-C are schematic illustrations of configurations of electrode assembly 22, in accordance with respective embodiments of the present invention. In the configuration shown in FIG. 2A, both electrode contacts 30 and 32 are configured to be in contact with muscle tissue 42, or to be at approximately equal distances from the tissue. For some applications, lead 26 comprises at least one fixation element 60, such as a screw-in fixation element, positioned along the lead between electrode contacts 30 and 32, so as to hold the lead in place with the electrode contacts both in contact with the tissue, or at approximately the same distance therefrom. Alternatively or additionally, the lead comprises a plurality of fixation elements in respective vicinities of the electrode contacts. As used in the present application, including in the claims, “screw-in fixation elements” include, but are not limited to, fixation elements that are shaped as screws, corkscrews, or helices.

In the configuration shown in FIG. 2B, lead 26 penetrates muscle tissue 42, such that both electrode contacts 30 and 32 penetrate the muscle tissue in a vicinity of fat pad 44, e.g., in contact therewith or within several millimeters therefrom.

In the configuration shown in FIG. 2C, electrode assembly 22 comprises a rotational-engagement fixation element 62, typically a screw-in fixation element. For some applications, fixation element 62 is sized such that its proximal end extends to the surface of the atrial wall when fully implanted, as shown in FIG. 2C, while for other applications, the fixation mechanism is shorter, such that its proximal end does not reach the surface of the atrial wall when fully implanted, but instead terminates inside the atrial wall. The surface of a proximal portion 64 of fixation element 62 is electrically insulated, e.g., comprises a non-conductive coating, such as Teflon or silicone, around a conductive core. A distal portion 66 of the fixation element is conductive, and serves as electrode contact 30 or electrode contact 32. As shown, proximal and distal portions 64 and 66 are coaxial, and the conductive core of proximal portion 64 is continuous with distal portion 66. Alternatively, electrode assembly 22 comprises another, non-rotational fixation element. For example, the fixation element may comprise straight electrode contacts, or flexible electrode contacts inserted via a sheath that is later withdrawn.

Insulated portion 64 is configured to be chronically disposed at least partially within atrial muscle tissue 42, and electrode contacts 30 and 32 are configured to be chronically disposed in contact with parasympathetic epicardial fat pad 44, typically within the fat pad. Optionally, a portion of insulated portion 64 penetrates into fat pad 44. Typically, but not necessarily, electrode contacts 30 and 32 are positioned entirely within the fat pad, such that no portion of the electrode contacts are in contact with atrial muscle tissue 42. A length of insulated portion 64 is typically greater than a thickness of the atrial wall, e.g., at least 1 mm or at least 2 mm. Avoidance of direct application of current to atrial muscle tissue 42 generally decreases the risk of undesired cardiac capture.

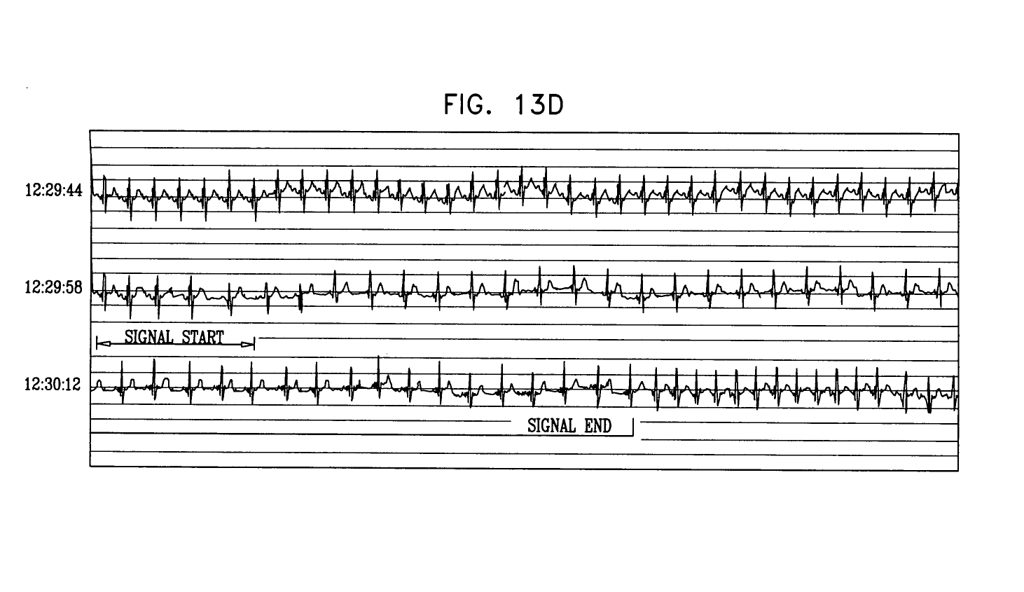

During implantation of the electrode assembly shown in FIG. 2C, distal portions of electrode contacts 30 and 32 are advanced entirely through and out the outward site of the cardiac muscle tissue of the atrial wall. The distal tips of electrode contacts 30 and 32 are typically positioned in fat pad 44. For some applications, during an implantation procedure, a check is performed to confirm that the distal portions of the electrode contacts have passed entirely through the cardiac muscle tissue, that the distal tips of the electrode contacts have entered the fat pad, and/or that the distal tips of the electrode contacts have not passed entirely through the fat pad and into the pericardial space. For some applications, techniques for monitoring such accurate positioning are used that are described hereinbelow with reference to FIG. 12. Typically, the distal tips of the electrode contacts are left in position outside the cardiac muscle of the atrial wall for at least one week. For some applications, electrode contacts 30 and 32 are inserted through atrial muscle tissue 42 until they are brought essentially entirely within fat pad 44. Thus the electrode contacts are positioned entirely within the fat pad, and outside the cardiac muscle.

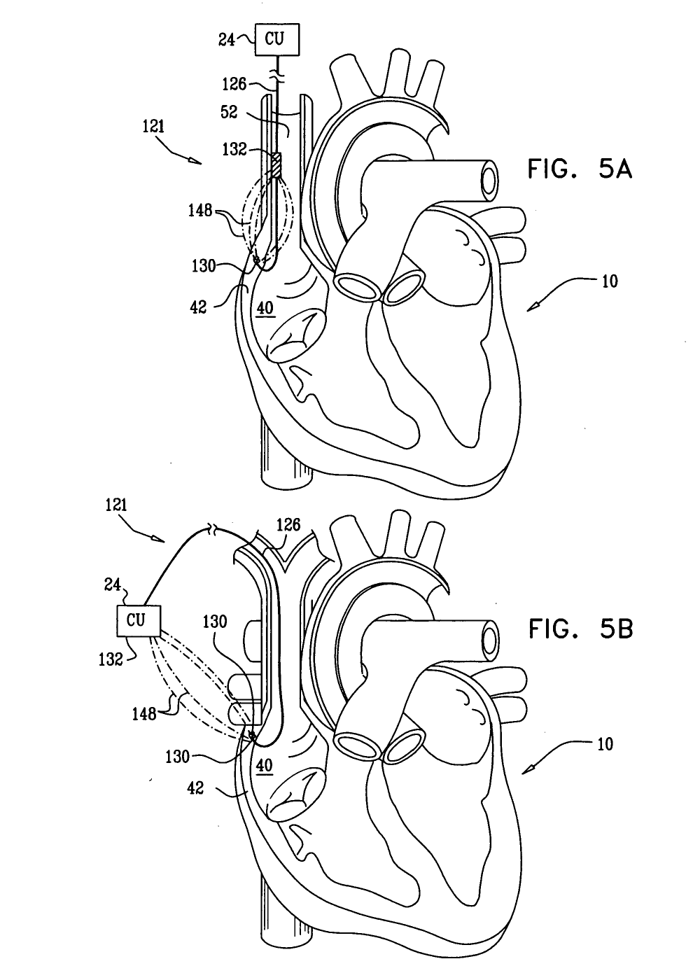

It is noted that although FIGS. 2A-C show two electrode contacts placed in the vicinity of fat pad 44 (e.g., acting as a cathode and an anode), the scope of the present invention includes using three or more electrode contacts placed in the vicinity of the fat pad (e.g., an anode between two cathodes, or a cathode between two anodes), or using one or more electrode contacts (e.g., one or more cathodes) placed in the vicinity, and another electrode contact disposed remotely from the fat pad (e.g., an anode). The remotely-disposed electrode contact, as appropriate, may be placed within a venous lumen of the subject, such as a coronary sinus (e.g., as described hereinbelow with reference to FIG. 5A, 5B, or 11), or at another site, or may be integrated into an outer conductive surface of control unit 24.

For some applications, electrode contact 30 is implanted in atrial muscle tissue 42 and/or in fat pad 44, while electrode contact 32 (e.g., serving as an anode) coupled to lead 26 remains in right atrium 40. For some applications, one or more additional electrode contacts (e.g., electrode contact 32) are also implanted in the atrial tissue and/or fat pad, and/or one or more additional proximal electrode contacts are provided on the lead of electrode contact 30.

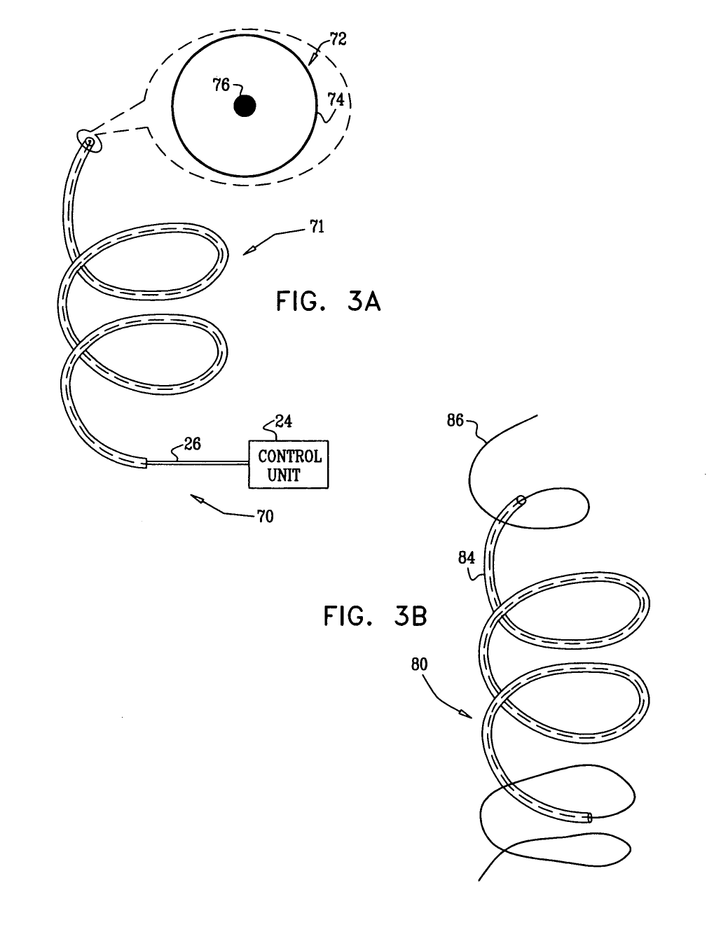

FIG. 3A is a schematic illustration of a screw-in electrode assembly 70 of system 20, in accordance with an embodiment of the present invention. Screw-in electrode assembly 70 comprises a screw-in fixation element 71 having at its distal tip a concentric bipolar electrode 72, and a lead 26. The enlarged portion of FIG. 3A shows a schematic cross-sectional view of bipolar electrode 72 of screw-in electrode assembly 70, in accordance with an embodiment of the present invention. Bipolar electrode 72 comprises an outer electrode contact 74 and an inner electrode contact 76, typically having opposite polarities. For example, outer electrode contact 74 may serve as an anode or a cathode. For some applications, outer electrode contact 74 extends along the entire length of screw-in fixation element 71 or a portion thereof, while for other applications the outer electrode contact is limited to only the distal tip of screw-in fixation element 71, in which case outer electrode contact 74 and inner electrode contact 76 are connected to lead 26 via separate wires.

FIG. 3B is a schematic illustration of a screw-in electrode assembly 80 of system 20, in accordance with an embodiment of the present invention. Screw-in electrode assembly 80 comprises a screw-in fixation element, which comprises an outer helical member 84 and an inner helical member 86. Outer helical member 84 is shaped so as to define a bore through the entire length of the member, and inner helical member 86 is shaped and sized so as to pass through the bore. All or a distal portion of inner helical member 86 is configured to serve as a first electrode contact, and all or a distal portion of outer helical member 84 is configured to serve as a second electrode contact.

During an implantation procedure, inner helical member 86 is typically rotated with respect to outer helical member 84 such that the inner helical member partially protrudes from the proximal end of the outer helical element, and substantially does not protrude from the distal end of the outer helical element (i.e., the end that enters the tissue first). After the outer helical element has been screwed into the tissue to a desired depth, the inner helical element is rotated within the outer helical element until the distal end of the inner helical element advances further into the tissue to a desired depth. For some applications, the distal end of the outer helical element is positioned within atrial muscle tissue 42, and the distal end of the inner helical element is positioned within parasympathetic epicardial fat pad 44, typically so that the electrode contact of the inner helical element is in direct electrical contact only with the fat pad, and not with the muscle tissue.

FIG. 3C is a schematic illustration of a screw-in electrode assembly 90 of system 20, in accordance with an embodiment of the present invention. Screw-in electrode assembly 90 comprises an outer helical fixation element 92 having a first radius, and an inner helical fixation element 94 having a second radius less than the first radius. The inner helical element is positioned within the outer helical element, such that the two helical elements are independently rotatable. All or a distal portion of inner helical member 92 is configured to serve as a first electrode contact, and all or a distal portion of outer helical member 94 is configured to serve as a second electrode contact.

During an implantation procedure, the inner and out helical members are independently rotated until each has been screwed into the tissue to a respective desired depth. The two helical members may be rotated together for a portion of the screwing. For some applications, the distal end of the outer helical element is positioned within atrial muscle tissue 42, and the distal end of the inner helical element is positioned within parasympathetic epicardial fat pad 44, typically so that the electrode contact of the inner helical element is in direct electrical contact only with the fat pad, and not with the muscle tissue. Alternatively, the distal end of the inner helical element is positioned within atrial muscle tissue 42, and the distal end of the outer helical element is positioned within parasympathetic epicardial fat pad 44, typically so that the electrode contact of the outer helical element is in direct electrical contact only with the fat pad, and not with the muscle tissue.

In an embodiment of the present invention, electrode assembly 22 comprises a first fixation element, which is configured for initial fixation during a first stage of an implantation procedure, and the electrode assembly comprises a second fixation element, which is configured to fix the electrode assembly in place during a second stage of the implantation procedure, with the aid of the initial fixation. For some applications, the first fixation element comprises a thin screw-in element, and the second fixation element comprises a screw-in element having a greater diameter. For some applications, the first fixation element is short and strong for fixation, and the second fixation element is longer and softer. For some applications, the electrode assembly does not comprises the second fixation element, and is held in place entirely or primarily by the first fixation element. For some applications, such electrode assemblies are used for pericardial implantation, while for other application, such electrode assemblies are used for epicardial implantation.

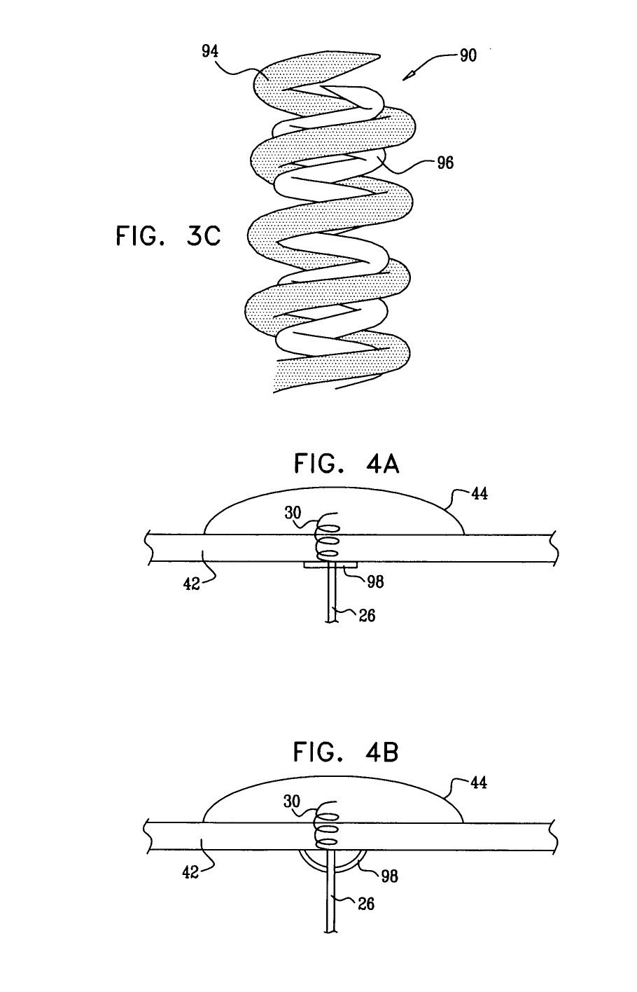

Reference is made to FIGS. 4A-B, which are schematic illustrations of electrode assemblies configured to minimize the risk of bleeding, in accordance with respective embodiments of the present invention. In embodiments of the present invention in which one or more of the electrode assemblies are configured such that at least a portion thereof penetrates the atrial wall and protrudes outside the atrium, the electrode assemblies are configured to minimize the risk of possible bleeding at the site of the penetration. For some applications, one or more of the following techniques are used to minimize this risk:
at least a portion of the electrode assembly that comes in contact with the surface of the cardiac wall has a greater cross-sectional area than a more distal portion of the electrode assembly adjacent thereto. For example, FIG. 4A shows a sealing element 98 having a cross-sectional area greater than that of lead 26 where the lead joins the sealing element. Sealing element 98 typically comprises a flexible material, such as silicone. For some applications, sealing element 98 is cupulate, such as shown in FIG. 4B;
a portion of the electrode assembly that comes in contact with the cardiac wall is configured to cause fibrosis (configuration not shown). For example, the portion may comprise a rough surface or a mesh, and/or may be coated with a drug for causing fibrosis.

FIG. 5A is a schematic illustration of a parasympathetic stimulation system 121, in accordance with an embodiment of the present invention. An electrode contact 130, e.g., part of a screw-in fixation element, is configured to implanted in atrial muscle tissue 42, either in a vicinity of SA node fat pad 46 (as shown in FIG. 5A), or in a vicinity of AV node fat pad 48 (configuration not shown). A second electrode contact 132 is disposed on a lead 126 which passes through superior vena cava 52, such that the second electrode contact is positioned in the superior vena cava. As shown in the figure, an electric field 148 is generated, the magnitude of which is highest in the area generally between electrode contacts 130 and 132. Specifically, a relatively high field strength develops in fat pad 44 (not visible in the figure) and at areas outside of heart 10, while a relatively low field strength develops in atrial muscle tissue 42 and the rest of the heart. In this manner, current generated between electrode contacts 130 and 132 affects fat pad 44 to a greater extent than muscle tissue 42. Alternatively, second electrode contact is placed in another blood vessel, such as an inferior vena cava, a coronary sinus, a right pulmonary vein, a left pulmonary vein, or a right ventricular base.

FIG. 5B is a schematic illustration of an alternative configuration of system 121, in accordance with another embodiment of the present invention. an electrode contact positioned outside of the heart and the circulatory system in a vicinity of the fat pad (but not in physical contact with the heart or the fat pad) serves as electrode contact 132 as described hereinabove with reference to FIG. 5A. For some applications, an outer surface of control unit 24 (the “can”) is conductive, and serves as this remote electrode contact. Alternatively, a separate electrode contact is provided for this purpose (configuration not shown). Aside from this difference, the embodiment of FIG. 5B is generally similar to that described with reference to FIG. 5A, with electrode contact 130 positioned in a vicinity of the fat pad. For some applications, electrode contact 130 uses a screw-in configuration. For some applications, control unit 24 is implanted on the lower right side of the subject's chest in a vicinity of heart 10. For some applications, electrode contact 130 is configured to be implanted subcutaneously. For example, the electrode contact may be implanted on the right side of the chest between the fourth and sixth ribs, typically in the vicinity of the left sides of the ribs, and/or under the ribs. In contrast, conventional pacemaker and ICDs cans are typically implanted on the upper left side of the subject's chest.

In an embodiment of the present invention, during an implantation procedure, the remote electrode contact is implanted before implanting electrode contact 130. Implanting electrode contact 130 comprises positioning electrode contact 130 at a plurality of locations of in the vicinity of the fat pad; while electrode contact 130 is positioned at each of the locations, driving a current between the remote electrode contact and electrode contact 130 and sensing a vagomimetic effect; and implanting electrode contact 130 such that it is positioned at the one of the locations at which a greatest vagomimetic effect was sensed.

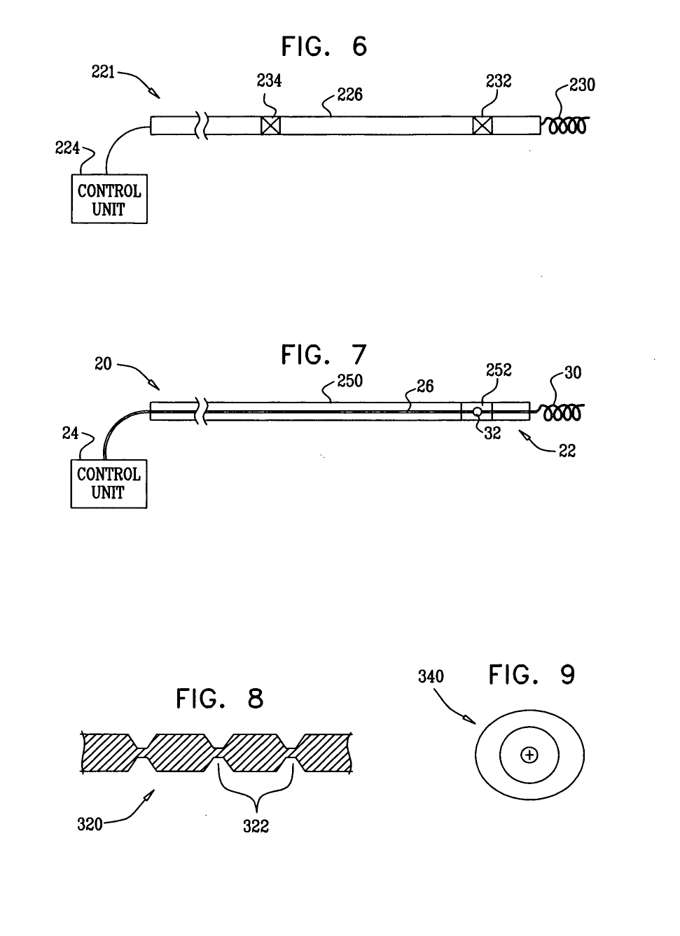

FIG. 6 is a schematic illustration of a parasympathetic stimulation system 221, in accordance with an embodiment of the present invention. A first electrode contact 230, e.g., part of a screw-in fixation element, is configured to be positioned in a lower portion of right atrium 40, typically in a vicinity of AV node fat pad 48. For some applications, first electrode contact 230 is configured to be implanted in atrial muscle tissue 42 in a vicinity of AV node fat pad 48. A second electrode contact 232 is disposed on a lead 226 which passes through superior vena cava 52, such that the second electrode contact is positioned in right atrium 40, typically near the first electrode contact, e.g., less than about 2 cm from the first electrode contact, but typically not in contact with the atrial wall. Alternatively, the second electrode contact is configured to be implanted in the atrial muscle tissue near the first electrode contact, such as using one of the electrode contact assemblies described hereinabove with reference to FIG. 2A-C or 3A-C. During stimulation of SA node fat pad 46 or AV node fat pad 48, a control unit 224 drives a current between first electrode contact 230 and second electrode contact 232, typically such that the first electrode contact functions as a cathode and the second electrode contact as an anode.

A third electrode contact 234 is disposed on lead 226 such that second electrode contact 232 is positioned between third electrode contact 234 and first electrode contact 230. The third electrode contact is positioned along the lead such that the third electrode contact is positioned in the superior vena cava, or in right atrium 40 in a vicinity of the superior vena cava.

Prior to driving the first and second electrode contacts to apply stimulation to the fat pad, control unit 224 uses the first and third electrode contacts, or the second and third electrode contacts, to sense the commencement of a P-wave. As soon as possible after detecting the P-wave (typically within 30 ms, such as within 10 ms), the control unit drives the first and second electrode contacts to stimulate the fat pad. Because the third electrode contact is positioned in the SVC (or near the SVC), the control unit is able to detect early depolarization of the upper portion of right atrium 40, and thus is able to detect the onset of the atrial depolarization early than would be possible using only the first and second electrode contacts. This enables the control unit to begin stimulation earlier in the atrial refractory period than would otherwise be possible. Such an earlier start typically allows the control unit to apply at least one more pulse during a burst of pulses than would otherwise be possible, without decreasing the interburst period of the pulses within the burst. Alternatively, the early detection of atrial depolarization enables the early detection of atrial capture that may result from the electrical stimulation.

For example, assume the atrial refractory period has a total duration of 100 ms, the pulse duration is 1 ms, pulse repetition interval (PRI) is 7 ms, circuitry of the control unit requires 30 ms to initiate stimulation after detection of the P-wave, and the P-wave arrives near the AV node fat fad about 30 ms after it is generated at the SA node. If the control unit were to detect the P-wave using the first and second electrode contacts, the control unit would have time to apply 10 pulses in the burst (pulses per trigger, or PPT). By instead using the first and third electrode contacts, the control unit is able to apply 14 pulses. This greater PPT enables an increased strength of stimulation without requiring an increase in other stimulation parameters, such as amplitude or pulse duration.

For some applications, system 221 comprises a fourth electrode contact positioned along lead 226, typically in a vicinity of second electrode contact 232 (configuration not shown). The control unit uses the third and fourth electrode contacts to sense the P-wave.

In an embodiment of the present invention, system 221 is integrated into an implantable cardioverter defibrillator (ICD), and third electrode contact 234 serves both for detecting the P-wave, as described above, and as the lead of the ICD conventionally positioned in superior vena cava 52.

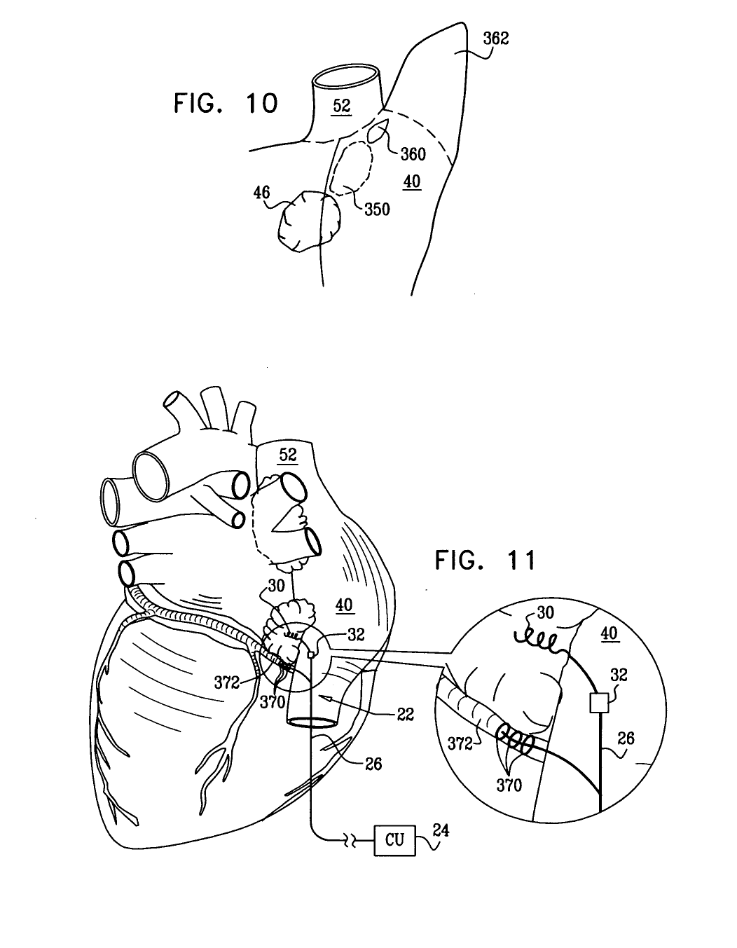

Reference is made to FIG. 7, which is a schematic illustration of a sheath 250, in accordance with an embodiment of the present invention. Sheath 250 is configured to enable stimulation of the target site during an implantation while at least one the electrode contacts (e.g., electrode contact 32) of electrode assembly 22 is still within the sheath. Sheath 250 includes at least one portion 252 through which electricity is conductible. For some applications, sheath 250 is shaped so as to define at least one window that defines the at least one portion 252. For other application, sheath 250 comprises a conductive element that serves as the at least one portion 252. For some applications, the sheath is configured such that the conducting portion extends from a distal opening of the sheath for at least 1 cm in a proximal direction along the sheath. For some applications, at least a portion of the sheath is deflectable, such as at least a portion of the conducting portion.

Before or during an implantation procedure, lead 26 is positioned in sheath 250 such that electrode contact 32 is aligned with conducting portion 252. During the implantation procedure, as a distal electrode contact, e.g., electrode contact 30, is positioned at various potential stimulation sites, system 20 applies stimulation between electrode contact 32 and electrode contact 30 within the sheath. For some applications, the sheath includes a plurality of conducting portions 252, and stimulation is applied sequentially through each of the portions and the proximal electrode.

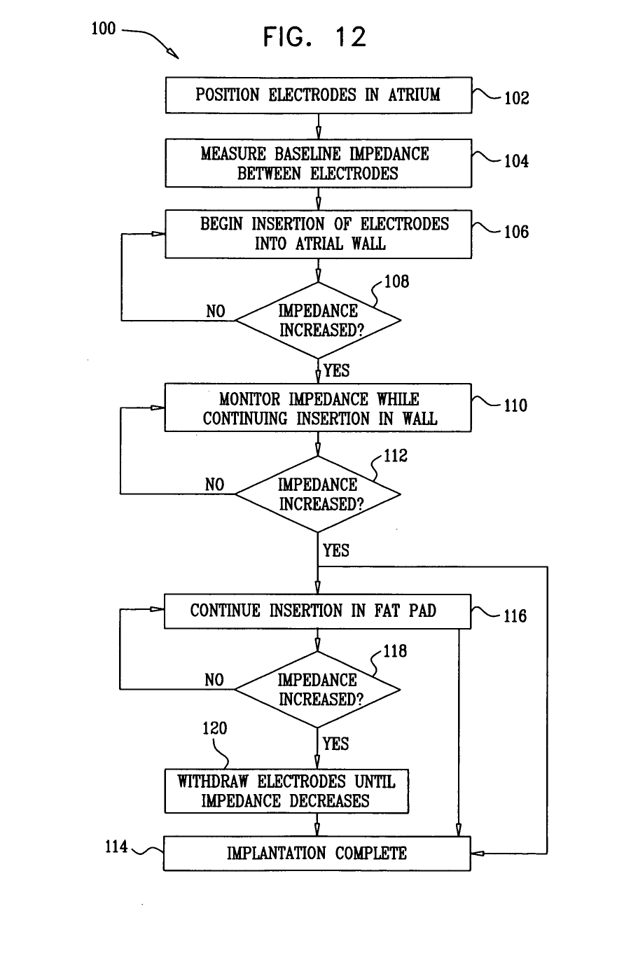

Reference is made to FIG. 8, which is a schematic illustration of an electrode lead 320 shaped so as to define grooves 322 on an external surface thereof, in accordance with an embodiment of the present invention. The grooved electrode lead is configured for trans-septum placement of an electrode contact at a left-atrial site. The grooves enable better sealing of the opening made in the septum for passage of the lead therethrough.

In some embodiments of the present invention, the stimulation site includes an interatrial groove. For example, the electrode contact may be placed at least partially in a vicinity of the groove, such as within about 2 mm of the groove, or in contact with the groove.

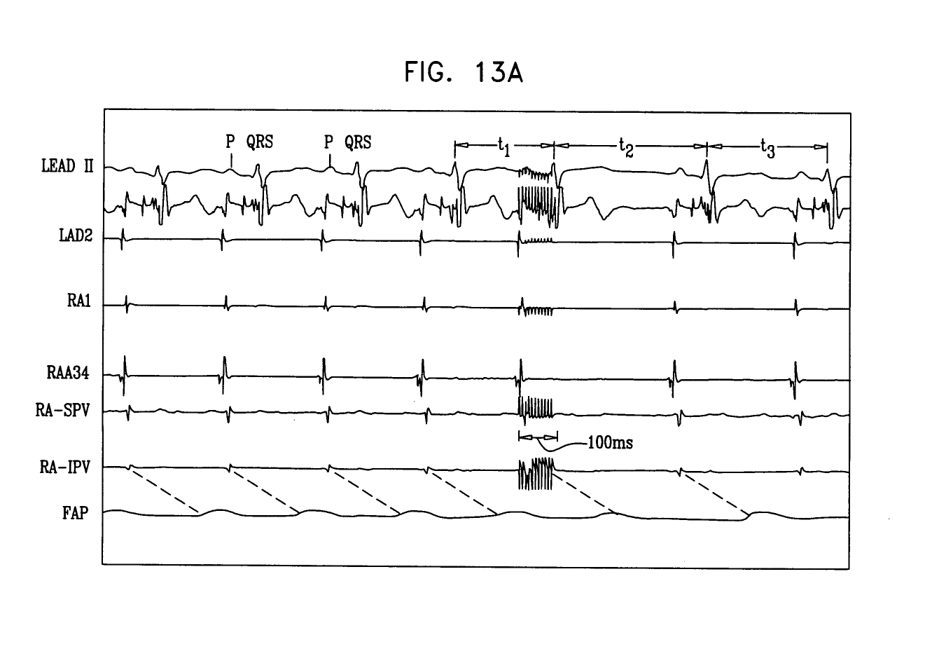

Reference is made to FIG. 9, which is a schematic illustration of tripolar ganglion plexus (GP) electrode assembly 340, in accordance with an embodiment of the present invention. This configuration generally limits the spread of any depolarization through the atrial tissue. The external anode generally blocks the propagation of atrial depolarization. The internal anode is used as a reference point, and as a means to limit the excitation potential of the external anode (known as an anode-induced virtual cathode), by dividing the current flow between the external and the internal anodes.

The stimulation waveform is typically quasi-trapezoidal, so as to avoid anodal break, for example. Furthermore, the stimulation waveform is typically asymmetrically balanced, with the discharge current spread over a longer period of time than the charging (stimulating) current. For example, for a stimulation with a duration of 0.5 ms, the discharge current may have a duration of at least 1.5 ms.

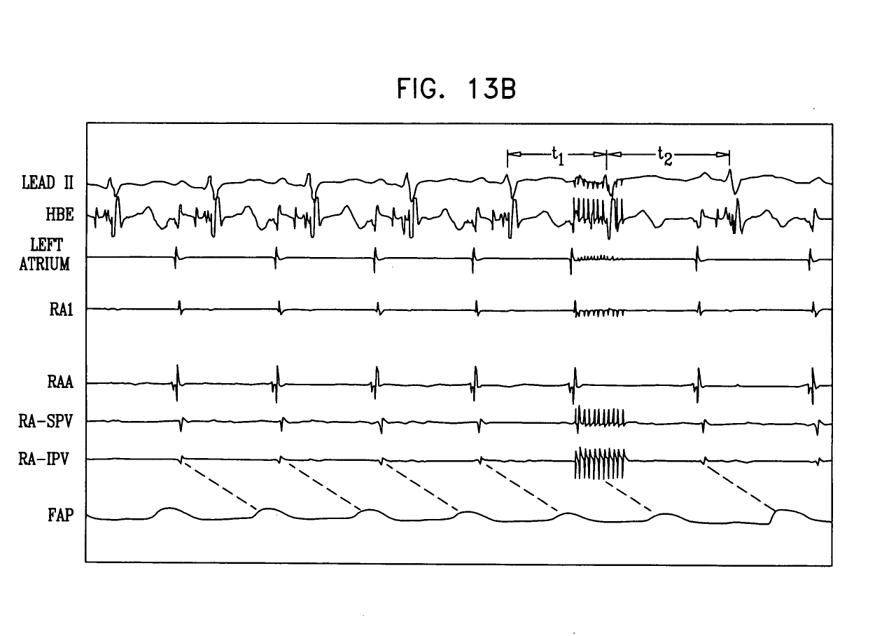

Reference is made to FIG. 10, which is a schematic illustration of an atrial region 350 for stimulation of postganglionic fibers, in accordance with an embodiment of the present invention. In this embodiment, at least one electrode contact is positioned at atrial region 350 within an atrium (typically the right atrium, or alternatively in the left atrium) in contact with the atrial wall, within the atrial wall, and/or through the atrial wall, in a vicinity of postganglionic fibers of a parasympathetic epicardial fat pad, such as SA node fat pad 46 and/or AV node fat pad 48, but not at or in the fat pad itself (i.e., not in contact with, or within, tissue of the cardiac wall that underlies the fat pad). Typically, but not necessarily, atrial region 350 is located generally between SA node fat pad 46 and an SA node 360, as shown in FIG. 10 (which also shows a right atrial appendage 362), or generally between AV node fat pad 48 and an AV node (location not shown). The inventors believe that stimulation of the postganglionic fibers in this region has a greater heart-rate-reduction effect than stimulation at or in the fat pads. The inventors also hypothesize that such postganglionic stimulation generally causes less afferent activation than stimulation of the fat pads or preganglionic fibers, and is thus less likely to cause side effects.

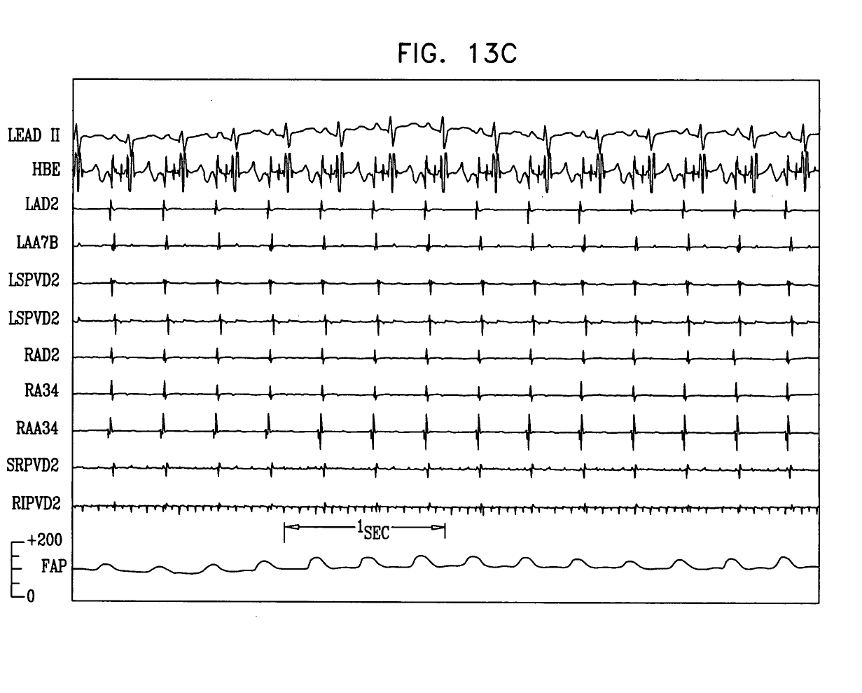

FIG. 11 is a schematic illustration of yet another configuration of stimulation system 20, in accordance with an embodiment of the present invention. In this embodiment, electrode assembly 22 comprises one or more electrode contacts 370 which are configured to be placed in a coronary sinus 372. For some applications, electrode contacts 370 comprise ring electrodes, as shown in the figure. Alternatively or additionally, electrode contacts 370 are incorporated into one or more baskets or coronary stents (configurations not shown). Electrode contact 30 (which may comprise any of the fixation elements described herein, such as a screw-in fixation element) is configured to be implanted in a vicinity of AV node fat pad 46, in contact with the atrial wall, within the atrial wall, and/or through the atrial wall into the fat pad. Optionally, electrode assembly also comprises an electrode contact 32 positioned along lead 26 in a vicinity of electrode contact 30. Control unit 24 is configured to drive a current between (a) electrode contact 30 and (b) one or more of electrode contacts 370, or, optionally electrode contact 32.

In an embodiment, control unit 24 is configured to drive the current in alternation between (a) electrode contact 30 and (b) each of electrode contacts 370 or, optionally, electrode contact 32. For some applications, the control unit configures electrode contact 30 to be the cathode, and the other contacts to be the anode. The alternation among electrode contacts 370 and 32 generally reduces the likelihood of exhausting the ganglia within AV node fat pad 46. Typically, the alternation has a frequency of between about 1 Hz and about 1000 Hz.

Alternatively, one or more of electrode contacts 370 are positioned in the inferior vena cava instead of or in addition to in coronary sinus 372. Further alternatively, electrode contact 30 is applied to another parasympathetic epicardial fat pad, such as the SA node fat pad, in which case electrode contacts are typically positioned in one or more of the right pulmonary arteries.

In an embodiment of the present invention, the burst length is configured to be as long as possible without extending beyond the conclusion of the AERP. To shorten the time from detection of an atrial depolarization to the initiation of the stimulation, the system charges the stimulation capacitor after each stimulation, and maintains the voltage on the stimulation capacitor until the next stimulation is applied, thus maintaining a “loaded and ready” situation. Each burst thus begins about 30 ms earlier than it otherwise could have if the capacitor had not been already charged, at the expense of battery life.

In an embodiment of the present invention, parasympathetic stimulation is combined with pacing at a location remote from the parasympathetic stimulation site. Application of such pacing allows the control unit to know exactly when the refractory period begins, in order to ensure that the parasympathetic stimulation is applied during the refractory period, and/or to apply the first pulse as early as possible during the refractory period, as described above. For some applications, the control unit applies the parasympathetic stimulation slightly before applying the pacing, such as up to 50 ms before the pacing, e.g., between about 10 ms and about 50 ms before the pacing. Alternatively, for some applications, the control unit applies the parasympathetic stimulation after a delay after application of the pacing, such as a delay equal to the estimated conduction time between the site of the pacing and the site of the parasympathetic stimulation.

In an embodiment of the present invention, system 20 comprises at least one electrode contact configured to be implanted epicardially (i.e., from outside the heart, rather than transvascularly). Control unit 24 drives the electrode contact to apply stimulation such that more of the current of the stimulation passes through pericardium than passes through myocardium. For some applications, such epicardial implantation is used for applying stimulation for preventing and/or terminating atrial fibrillation, typically by applying the stimulation to the AV node fat pad. For some applications, techniques are used that are described in U.S. patent application Ser. No. 11/657,784, filed Jan. 24, 2007 and/or U.S. patent application Ser. No. 10/560,654, filed May 1, 2006, both of which are assigned to the assignee of the present application and are incorporated herein by reference. For some applications, such epicardial implantation is used for treating a subject suffering from both heart failure and atrial fibrillation. For some applications, such stimulation is applied only when a sensed heart rate of the subject exceeds a threshold heart rate, such as about 60 BMP.

In an embodiment of the present invention, a parasympathetic epicardial fat pad radiopaque marker is placed during an open chest operation, to facilitate later positioning (e.g., position and/or angle of penetration) of an intra-atrial electrode contact.

In an embodiment of the present invention, system 20 is configured to detect whether the electrode contact has become dislodged and passed out of the atrium into the ventricle (either from the right atrium to the right ventricle, or the left atrium to the left ventricle). Application of the stimulation to the ventricle may cause ventricular capture, which could be potentially dangerous. Control unit 24 uses the electrode contact to sense a local electrogram. The control unit analyzes the electrogram to determine whether it is characteristic of atrial electrical activity. Atrial signals have characteristic signatures, such as shape and signal width, that are different from those of ventricle signals, both when the subject is in NSR and in AF, as is known to those skilled in the art. Upon finding that the electrode contact remains at its implantation site in the atrium, the control unit applies parasympathetic stimulation, such as using techniques described herein. Otherwise, the control unit withholds applying the stimulation. Typically, the control unit configures the stimulation to avoid causing capture, such as by setting the signal strength to be too low to cause capture, applying the signal during the atrial refractory period, or using other techniques for avoiding capture described herein.

For some applications, the control unit performs this verification of atrial location generally continuously for application of stimulation. Alternatively, the control unit performs the verification periodically, such as once per minute, or once per hour. For some applications, the control unit periodically withholds applying the parasympathetic stimulation during a sensing period having a duration of at least 2 seconds, e.g., at least 5 seconds, or at least one minute, and performs the sensing during this period. Providing such a non-stimulation period generally provides a cleaner sensed signal because the parasympathetic stimulation is less likely to cause interference. For some applications, the control unit uses known signatures of atrial activity, while for other applications, the control unit performs a characterization of the subject's atrial electrical activity to generate a unique signal fingerprint for the subject, during a calibration procedure performed prior to, during, or soon after implantation of the electrode contact.

For some applications, system 20 includes a ventricular lead configured to be placed in a ventricle. The control unit periodically compares the signal detected by the electrode contact in the atrium and the signal sensed by the ventricular lead in the ventricle. The control unit interprets a change in the comparison as indicating that the electrode contact or the lead has moved, and thus withholds driving the electrode contact to apply the parasympathetic stimulation.

Reference is made to FIG. 12, which is a flow chart schematically illustrating a method 100 for implanting an electrode contact at a desired position in parasympathetic epicardial fat pad 44, in accordance with an embodiment of the present invention. For some applications, method 100 is used to implant electrode contact 30 and/or electrode contact 32, described hereinabove with reference to FIG. 1 and FIGS. 2A-C; the screw-in electrode assemblies described hereinabove with reference to FIGS. 3A-C; or any of the other electrode assemblies described herein or otherwise known in the art.

Method 100 aids in the positioning of the distal portion of one or more electrode contacts in parasympathetic epicardial fat pad 44, which for many applications is the optimal positioning. It is generally desirable not to advance the electrode contact entirely through the fat pad and out into the pericardial space, where the presence of an electrode contact may cause pericardial irritation and effusion.

Method 100 begins with the positioning of the electrode contacts in an atrium, such as right atrium 40 or a left atrium, in a vicinity of parasympathetic epicardial fat pad 44, at an electrode contact positioning step 102. During the implantation procedure, impedance between the electrode contacts is periodically or generally continuously monitored to aid with locating and fixating the electrode contacts in the fat pad. Such monitoring is generally achieved by passing a fixed current pulse between the electrode contacts and measuring the required voltage, or by applying a fixed voltage and measuring the resulting current. Such pulses are typically applied at least once every two seconds, to provide a generally continuous impedance assessment. At a baseline impedance measurement step 104, baseline impedance is measured while the electrode contacts are still in the atrium. Such impedance is relatively low while the electrode contacts are in contact only with blood.

Insertion of the electrode contacts into atrial muscle tissue 42 begins at a insertion step 106. Impedance is monitored during the insertion, and at an impedance check step 108, it is determined whether an increase in impedance has occurred. Such an increase indicates that insertion into the muscle tissue has begun. Further insertion of the electrode contacts through the muscle tissue while monitoring impedance continues at a continuing insertion step 110.

At an impedance check step 112, it is determined whether a further increase in impedance has occurred. Such a further increase indicates that the electrode contacts have entered fatty tissue of fat pad 44. Optionally, upon detecting this further increase in impedance, insertion is stopped immediately, at an implantation completion step 114. Alternatively, the electrode contacts are advanced slightly more into the fat pad in order to provide better contact, and a continued insertion step 116. Impedance is monitored, and at an impedance check step 118, it is determined whether impedance has decreased. Such a decrease indicates that the electrode contacts have been inserted too far, and have exited the fat pad into the pericardial space. The electrode contacts are thus withdrawn while monitoring impedance until the impedance returns to approximately its previous level, at an electrode contact withdrawal step 120, upon which the implantation is complete at step 114.

For some applications of method 100, two or more electrode contacts are positioned in the fat pad, and impedance is monitored between or among the electrode contacts. For other applications, one or more electrode contacts are positioned in the fat pad, and impedance is monitored between each of the electrode contacts and one or more electrode contacts positioned in the atrium.

In an another embodiment of the present invention, method 100 monitors pressure instead of impedance. The pressure at or near the distal tip of the one or more electrode contacts, or of one or more dedicated guidewires, is monitored during the positioning of the electrode contacts. An increase in pressure detected at check step 108 indicates that the electrode contacts have entered the atrial wall. A decrease in pressure detected at check step 112, characterized by sinusoidal periodic changes in pressure, indicates penetration of the electrode contacts into the fat pad tissue. If the distal tip is undesirably further advanced into the pericardial space, a flat or spiked pressure pattern is observed at impedance check step 118. Corrections to electrode contact position are made accordingly as to position the electrode contact within the fat pad tissue but not in the pericardial space, at withdrawal step 120.

In another embodiment of the present invention, the positioning of the one or more electrode contacts is achieved under echocardiogram visualization, such as transesophageal echocardiogram visualization, or transthoracic echocardiogram visualization. The active fixation screw is advanced through the atrial wall, but not into the pericardial space.

In an embodiment of the present invention, the one or more electrode contacts are driven to apply parasympathetic stimulation while being advanced into the cardiac muscle and/or fat pad (the stimulation is typically applied either at subthreshold strength, and/or only during the AERPs of each cardiac cycle). Advancement of the electrode contacts is stopped when a desired heart-rate-lowering effect of stimulation is observed, so that the electrode contact is not advanced further than needed. If an insufficient heart-rate-lowering effect is observed at all depths of insertion, the electrode contacts are withdrawn and re-inserted in at a slightly different position and/or angle.

In an embodiment of the present invention, the control unit is configured to drive the electrode contacts to apply to the fat pad a burst of pulses comprising one or more initial pulses followed by one or more subsequent pulses. The control unit sets a strength of the initial pulses to be insufficient to cause parasympathetic activation of the fat pad, and a strength of the subsequent pulses to be sufficient to cause the parasympathetic activation. The initial pulses serve to precondition the fat pad for more effective subsequent parasympathetic activation. For some applications, the strength of the initial and subsequent pulses are set during a calibration procedure, in which the electrode contacts are driven to apply a plurality of calibration bursts at respective calibration strengths, whether the calibration bursts cause a vagomimetic effect is sensed, a minimal strength necessary to cause the vagomimetic effect is found, and the preconditioning strength of the initial pulses is set to be less than the minimal strength, and the activating strength of the subsequent pulses to be at least the minimal strength.

In an embodiment of the present invention, system 20 comprises at least two electrodes contacts that are configured to be positioned intra-atrially at least two separate vagomimetic sites (i.e., sites causing a vagal response when stimulated), or at least the electrode contacts that are configured to be positioned at least three separate vagomimetic sites. For some applications, a first one of the electrode contacts is positioned in a vicinity of SA node fat pad 46, and a second one of the electrode contacts AV node fat pad 48. For some applications, one or more (such as all) of the electrode contacts are coupled to the wall using a screw-in fixation element.

In an embodiment, control unit 24 is configured to simultaneously drive all of the electrode contacts to apply stimulation to the respective sites. For some applications, the control unit is configured to drive the electrode contacts to apply the stimulation during the atrial absolute refractory period.

In an embodiment, control unit 24 is configured to drive each of the electrode contacts to apply stimulation to its respective site during a local refractory period at the site. For some applications, the control unit uses each of the electrode contacts to both apply the stimulation and to sense a local electrogram, which the control unit uses to detect the local refractory period.

In an embodiment of the present invention, combined intra-atrial stimulation of the SA and AV fat pad nodes is applied to treat a subject suffering from heart failure and paroxysmal atrial fibrillation (AF). According to one method for such treatment, control unit 24 detects whether the subject is currently in normal sinus rhythm (NSR) or experiencing an episode of AF. If the subject is experiencing the episode of AF, the control unit drives the electrode contact in the vicinity of the AV node fat pad to apply stimulation to AV node fat pad, in order to reduce the ventricular rate (stimulation of the SA node fat pad has minimal effect on ventricular rate during AF). If, on the other hand, the subject is in NSR, the control unit drives the electrode contact in the vicinity of the SA node fat pad to apply stimulation to the SA node fat pad, in order to reduce the ventricular rate. For some applications, if the subject is in NSR, the control unit measures the heart rate and compares it to a threshold value (e.g., between about 60 and about 150 BPM, such as about 80). The control unit drives the electrode contact in the vicinity of the SA node fat pad to apply the stimulation only if the heart rate is greater than the threshold.

In an embodiment of the present invention, system 20 comprises a sensor of cardiac activity, configure to generate a cardiac activity signal. Control unit 24 is configured to receive and analyze the signal, and, upon finding that the subject is in AF, to perform cardioversion by applying simultaneous stimulation of the AV node and SA node fat pads. For some applications, this embodiment is performed in combination with techniques described in U.S. application Ser. No. 11/724,899, filed Mar. 16, 2007, entitled, “Parasympathetic stimulation for termination of non-sinus atrial tachycardia,” which is assigned to the assignee of the present application and is incorporated herein by reference.

In an embodiment of the present invention, a method is provided for combined reduction of heart rate and prolongation of PR interval to obtain optimal cardiac performance, comprising: (a) intra-atrially stimulating the SA node fat pad to cause heart rate reduction, (b) intra-atrially stimulating the AV node fat pad to cause PR prolongation, (c) sensing a measure of cardiac performance (e.g., cardiac contractility, blood pressure, or cardiac output), and (d) responsively to the measure, configuring one or more parameters of the stimulation of the AV node fat pad to improve the sensed measure of cardiac performance.

In an embodiment of the present invention, a method is provided for achieving cardiac arrest, comprising: intra-atrially applying stimulation to both the SA node and AV node fat pads, and configuring the stimulation to achieve the cardiac arrest. This method is typically performed during a cardiac surgical procedure.

In an embodiment of the present invention, a method is provided for delivering rate control therapy while maintaining AF. The control unit detects whether the subject is in AF. When the control unit finds that the subject is in AF, the control unit drives the electrode contact to apply stimulation to the AV node fat pad, and configures the stimulation to reduce the heart rate. When, on the other hand, the control unit finds that the subject is in NSR, the control unit drives the same electrode contact or another electrode contact to apply a pacing signal to the atrium, and configures a rate of the signal to be at least 1.5 Hz (e.g., at least 20 Hz) in order to convert the subject to AF. For some applications, the pacing signal is applied at the same site as the stimulation of the AV node fat pad, for example using at least one common electrode, or, alternatively, using different electrodes.

Such AF maintenance generally reduces the frequency of recurring transitions between AF and NSR, which transitions are common in subjects with AF, particularly in subjects with chronic episodic AF. Such repeated transitions are generally undesirable because: (a) they often cause discomfort for the subject, (b) they may increase the risk of thromboembolic events, and (c) they often make prescribing an appropriate drug regimen difficult. Drug regimens that are beneficial for the subject when in AF are often inappropriate when the subject is in NSR, and vice versa. Knowledge that the subject will generally remain in AF typically helps a physician prescribe a more appropriate and/or lower-dosage drug regimen.

In some embodiments of the present invention, a subject is identified as suffering from a cardiac condition, and intra-atrial stimulation of one or more parasympathetic epicardial fat pads is applied to treat the condition. The condition typically includes chronic heart failure (HF), atrial flutter, chronic atrial fibrillation (AF), chronic AF combined with HF, atrial flutter combined with HF, hypertension, angina pectoris, and/or an inflammatory condition of the heart. Alternatively or additionally, the stimulation is applied to regular the production of nitric oxide (NO) (e.g., by changing the level of at least one NO synthase, e.g., increase a level of eNOS), such as in combination with techniques described in U.S. application Ser. No. 11/234,877, filed Sep. 22, 2005, entitled, “Selective nerve fiber stimulation,” which is assigned to the assignee of the present application and is incorporated herein by reference.

For some applications, the stimulation is configured to stimulate vagal ganglion plexuses (GPs). In other embodiments, the stimulation is applied at a site in the pulmonary veins of the subject, or in the great veins leading to the right atria (vena cava veins and coronary sinus).

In an embodiment of the present invention, stimulation of autonomic sites in heart failure subjects has a therapeutic effect by multiple mechanisms of action, including, but not limited to, control over heart rate, increase in coronary blood flow, attenuation of inflammation and apoptosis, reduction in wall tension, and improved relaxation.

The application of chronic autonomic system stimulation using an intra-atrial electrode for treating heart failure subjects enables separate control of rate (by stimulation of the SA node fat pad) and conduction time (by stimulation of the AV node fat pad). Other autonomic stimulation methods generally have an effect on both rate and conduction time. Furthermore, the implantation of atrial electrodes in heart failure subjects has become recently become more common. The autonomic stimulation techniques described herein can generally be applied using the same atrial electrodes implanted for other purposes, and thus may not require the performance of a separate implantation procedure. In addition, the stimulation of parasympathetic epicardial fat pads, the ganglion plexus (GP), and/or postganglionic fibers is believed by the inventors to cause less afferent activation than stimulation of preganglionic axons, and thus fewer side effects. Also, procedures to implant intra-atrial electrodes generally do not require general anesthesia.

For some applications, the intra-atrial stimulation techniques described herein are used in combination with other techniques for treatment of heart failure known in the art, such as techniques that use cervical and thoracic vagal stimulation, intravascular vagal stimulation, and/or epicardial implantation of electrodes for fat pad stimulation.

In an embodiment of the present invention, the intra-atrial fat pad stimulation techniques described herein are used for treating subjects that suffer from both heart failure (HF) and concurrent atrial fibrillation (AF). In such subjects, the risk of causing inadvertent atrial excitation is minimized, since the atria are fibrillating and cannot generally be excited. For some applications, for the treatment of subjects suffering from both HF and AF, SA node fat pad stimulation is applied alone or in conjunction with AV node fat pad stimulation. For some applications and subjects, when SA node or AV node fat pad stimulation is applied in subjects suffering from both HF and AF, the stimulation elicits the beneficial effects of heart failure therapy, and at the same time delivers the beneficial effects of AF prevention, such as preventing remodeling of the atria and reducing atrial wall tension, such as described in above-mentioned U.S. patent application Ser. No. 11/657,784, filed Jan. 24, 2007 and/or U.S. patent application Ser. No. 10/560,654, filed May 1, 2006. For some applications, the SA node fat pad alone, the AV node fat pad alone, or both fat pads are stimulated to treat heart failure subjects with AF even if the stimulation has no or only a minimal effect on the heart rate. (Control over heart rate is usually not achieved when the SA node fat pad is stimulated alone.)

In some embodiments of the present invention, the control unit is configured to apply the stimulation in a series of bursts, each of which includes one or more pulses. For some applications, the control unit is configured to apply one burst per cardiac cycle, synchronized with a feature of the cardiac cycle of the subject. For example, each of the bursts may commence upon detection of a P-wave, or after a delay after a detected R-wave, P-wave, or other feature of an ECG. Alternatively, for some applications, the control unit is configured to synchronize the bursts with other physiological activity of the subject, such as respiration, muscle contractions, or spontaneous nerve activity. Further alternatively, for some applications, the bursts are not synchronized with the cardiac cycle, or with other physiological activity.

In an embodiment of the present invention, the control unit configures the stimulation such that at least one pulse in each burst is applied during the atrial effective refractory period (AERP), such as at least half of the pulses in each burst, or all of the pulses in each burst. For some applications, each burst is initiated upon detection of a naturally-occurring P-wave (e.g., immediately upon detection of the P-wave, or within 1-150 ms of detection of the P-wave, and is applied entirely within the AERP.

In some embodiments of the present invention, the techniques described herein are used for treatment of heart failure and/or atrial fibrillation. Alternatively or additionally, the techniques described herein are used post-myocardial infarct, post heart surgery, post heart transplant, during heart surgery, or during an otherwise indicated catheterization (such as PTCA).

Alternatively or additionally, the techniques described herein are used for classic pacing indications (e.g., bradycardia, sick sinus syndrome, or cardiac resynchronization), where instead of applying a single pacing signal, the device applies a burst of pulses, each burst having a duration that is shorter than the AERP, such as no more than 85% of the AERP. Typically the first pulse of each burst paces the atrium, and the subsequent pulses generate a vagomimetic response, but do not cause additional capture, because they are applied during the AERP.

In some embodiments of the present invention, the apparatus is configured to pace the atria, in addition to applying parasympathetic stimulation. The apparatus is configured to begin application of each of the stimulation bursts when the atria is not refractory, and thus the first one or more pulses (e.g., the first one) of the burst causes atrial depolarization, and the remaining pulses of the burst, falling within the effective refractory period, facilitate the vagomimetic effect.

In some embodiments, the first pulse in the train, which is configured to cause atrial depolarization, has an amplitude or pulse duration that is greater than that of the subsequent pulses. For these embodiments, the rate and timing of the stimulation bursts are set according to the clinical indication for atrial pacing, i.e., according to the desired heart rate. Such indications include, but are not limited to, bradycardia and sick sinus syndrome.

In some embodiments of the present invention, the system further comprises a pacemaker/CRT and/or an ICD. For some applications, the control unit is configured to apply the vagomimetic stimulation in bursts including one or more pulses. For some applications, the control unit synchronizes the bursts with a feature of the cardiac cycle. For some applications, the control unit sets a duration of each of the bursts to be no longer then the atrial effective refractory period at the site of stimulation.

In some embodiments of the present invention, the apparatus comprises a CRT pacemaker-like lead that is positioned at a coronary sinus. The electrode comprises a bipolar stimulation tip, that is advanced along the cardiac veins until the stimulation tip is positioned over the left ventricle, and additional proximal bipolar stimulation rings that are positioned in the coronary sinus. The distance between the two electrode sets is typically between about 2 to about 10 cm. The control unit drives the second, proximal, electrodes to stimulate the local vagomimetic site. This site is stimulated after the distal electrode set is stimulated and ventricular depolarization is initiated. Thus, the vagomimetic stimulation does not to interfere with the CRT signal propagation and ventricular depolarization sequence, even if local cardiac excitation occurs, since it is timed to the wanted timing for excitation of the underlying cardiac tissue. Alternatively, vagomimetic stimulation is applied before the distal signal, according to the desired AV delay and interventricular delay.

In some embodiments of the present invention, a method for pacing a heart comprises delivering a burst of pulses, wherein the burst duration is no longer then the effective refractory period at the site of the pacing. For some applications, this method is used for pacing an atrial site, while for other applications, this method is used for pacing a non-atrial site, such as a ventricular site.

In an embodiment of the present invention, an additional sensing and/or pacing electrode is placed in the right ventricle. For some applications, this electrode is used to pace the heart if the heart rate falls below a certain threshold. For other embodiments, the ventricular electrode is used to sense the ventricular rate for confirmation of the sensing of the P-wave by the atrial electrode. In practice, a depolarization sensed in the ventricle inhibits the detection of a P-wave and/or the stimulation by the atrial electrode, for a period of about 250 ms. Thus, ventricular premature beats that might be accidentally detected also in the atria are not detected, and stimulation outside of the refractory period is avoided.

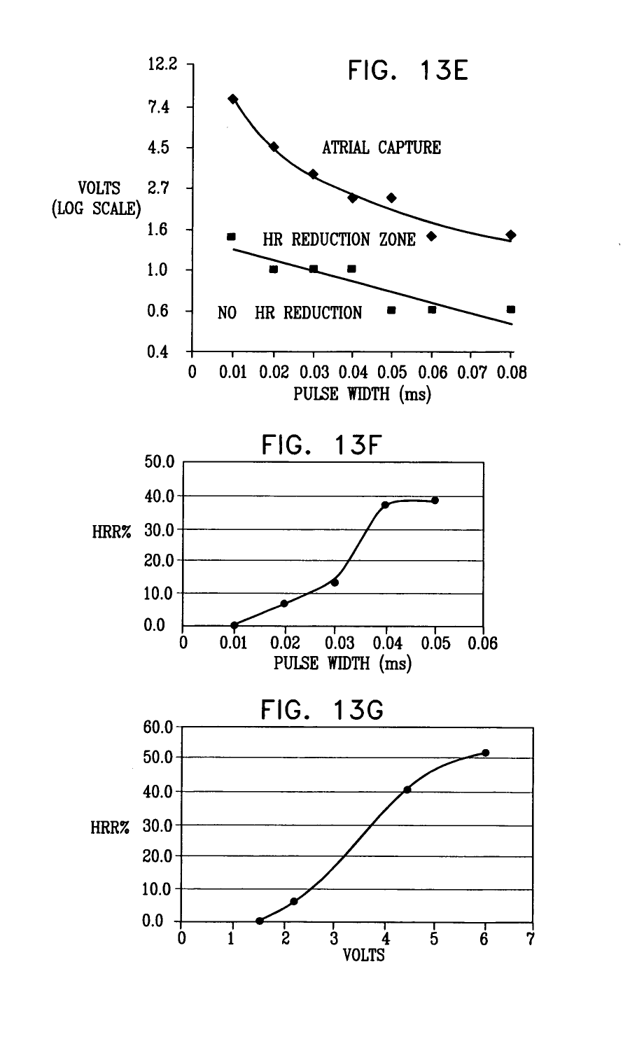

Reference is now made to FIGS. 13A-G, which are graphs showing data recorded in a dog experiment performed in accordance with an embodiment of the present invention. A first intra-atrial active fixation lead was implanted, penetrating through the atrial wall from within the right atrium, to arrive at the vicinity of the sinoatrial (SA) node fat pad, and a second intra-atrial activate fixation lead was implanted in the vicinity of the atrioventricular (AV) node fat pad. Stimulation of the SA and AV node fat pads was achieved using intra-atrial electrode contacts suitable for chronic implantation, in an appropriate medical procedure to chronically implant the electrode contact. Following the experiment, the dog was sacrificed.

Two small canines were utilized (15-20 kg), such that the size of heart chambers and musculature dimensions were approximately 60% of adult human size. Choice of this model enabled the use of ‘off-the-shelf’ electrode contacts marketed for human use, without any adaptations. (For adult human use, some embodiments of the present invention utilize larger electrode contacts than are provided for normal pacing applications.)

Bilateral thoracotomy was performed under general anesthesia using phenobarbital, and the animals were mechanically ventilated. Recording electrodes were placed as described hereinbelow, and direct visualization of the epicardial surfaces was achieved. The pericardium was opened, and recording electrodes were placed on the left atrial roof, left atrial appendage, left superior pulmonary vein, left inferior pulmonary vein, right superior pulmonary vein, and right inferior pulmonary vein. All “vein” electrodes were placed externally to and in contact with the respective vein.

An active fixation lead with a deflectable sheath was used to facilitate placement of the electrode contact at the SA node fat pad. The tip of the sheath was directly viewed, to facilitate placement of the electrode contact. The electrode tip was placed in an open surgical procedure into the right external jugular vein, then passed into the right atrium, midway between the superior vena cava and the inferior vena cava (IVC). The deflectable sheath was deflected toward the free wall (i.e., in an upward direction, since the animal was lying on its left side). The electrode contact was advanced in the dorsal direction, toward the interatrial septum, and was screwed into the muscular part of the right atrium, at a location that was approximately midway between (a) the meeting point of the interatrial septum with the atrial free wall and (b) the crista terminalis, in the vicinity of the SA node fat pad.

In an embodiment of the present invention, all or a portion of this implantation technique is used in a human subject for chronically implanting an electrode contact in a vicinity of a SA node fat pad from within an atrium. For some applications, the electrode contact is inserted into the atrial musculature at a posterior portion of the atrium within about one cm of the interatrial septum. Alternatively, the sheath is pre-shaped. Typically, the sheath is more rigid than the electrode contact. For some applications, the sheath presses (at least in part) against the IVC alternatively or additionally to pressing against the free atrial wall. The applied pressure helps fixate the electrode contact. The sheath also typically helps position the electrode contact at a desired orientation, position, or angle with respect to the tissue to which the electrode contact is fixated.

In order to place an electrode contact at the atrioventricular (AV) fat pad, a second electrode contact was advanced from the right external jugular vein toward the right atrium, until it reached the area of insertion of the inferior vena cava (IVC) into the right atrium. Once the second electrode contact was placed at the most caudal point of the insertion of the IVC, the electrode contact was further advanced approximately 1 cm. The sheath was then deflected and directed towards the dorsolateral wall of the atrium. The inferior atrial wall was then pushed to a perpendicular position in relation to the IVC axis, and pushed towards the electrode contact. The second electrode contact was then screwed through the atrial musculature to the vicinity of the AV node fat pad. In this manner, the second electrode contact was positioned in a vicinity of the AV node fat pad. Data collected during stimulation of the AV node fat pad showed that stimulation of the AV node fat pad also reduced heart rate (data not shown).

In an embodiment of the present invention, all or a portion of this implantation technique is used in a human subject for chronically implanting an electrode contact in a vicinity of an AV node fat pad from within an atrium.

Activation of the SA node fat pads was achieved with signals that were non-excitatory to the atrial muscle tissue, using two different methods. The data shown in FIGS. 13A-G relate to stimulation of the SA node fat pad.

### Atrial Effective Refractory Period (AERP) Stimulation

A human grade muscle stimulator applied symmetric biphasic current pulses to the fat pads (SA node and AV node fat pads) A short burst of pulses was applied within the atrial refractory period, once every several beats, resulting in substantial cycle prolongation. AERP stimulation of the SA node fat pad, which produced the results shown in FIGS. 13A and 13B, was limited to within the effective refractory period. Atrial capture was observed when stimulation extended beyond this period (e.g., for bursts lasting longer than about 130 ms). However, the absolute atrial refractory period was actually shorter than the pulse bursts applied, as demonstrated by applying a burst of relatively long 1.5 ms pulses, where atrial capture could be observed even when the stimulation period was limited to 40 ms. Additionally, 0.02 ms pulses applied outside of the effective refractory period were sufficient to cause capture (data not shown).

Exemplary parameters that produced heart rate reduction included: pulses per trigger (PPT, i.e., pulses applied in one cardiac cycle)=11, pulse repetition interval (PRI)=10 ms and 15 ms, pulse width=0.02-1 ms, current=5-20 mA (e.g., 8 mA). Parameters such as these yielded a heart rate reduction (HRR) from 128 to 95 BPM.

### Asynchronous Stimulation

Stimulation by applying monophasic voltage pulses was performed, without synchronizing the stimulation to the cardiac cycle. Pulse width was manipulated to achieve effective fat pad (SA node and AV node fat pads) stimulation and to avoid atrial capture. In addition, the pulse voltage was also shown to control these effects.

Exemplary parameters that caused heart rate reduction (e.g., from 196 to 160 BPM) were reached while still maintaining a good therapeutic window, i.e., a substantial difference between the minimum voltage that yielded atrial capture and the minimum voltage that yielded effective fat pad stimulation, i.e., heart rate reduction (see FIG. 13E). Heart rate reduction was achieved, for example, using 1.5-8 V (e.g., 2.4 V), 0.01-0.08 ms (e.g., 0.04 ms) pulse width, and 5-20 Hz (e.g., 20 Hz).

Heart rate reduction was found to be correlated with both pulse width and voltage of stimulation, as shown in FIGS. 13F and 13G.

FIG. 13A is a graph showing data recorded in accordance with the AERP stimulation method described hereinabove, in accordance with an embodiment of the present invention. Pulse width was set to 8 mA, PRI was 10 ms, and PPT was 10. Sensing electrodes measured electrical activity on the skin surface (Lead II), at the His bundle, left atrial roof (LAD2), at the right atrial roof (RA1), right atrial appendage (RAA), right superior pulmonary vein (RA-SPV), inferior pulmonary vein (RA-IPV). Femoral arterial pressure (FAP) was also measured.

Dashed lines are shown linking P-waves on the RA-IPV data line with the corresponding pressure pulse on the FAP data line, although it is noted that the pressure pulse is actually caused by the QRS-complex, shown most clearly on the LEAD II data line.

Four normal cardiac cycles are shown in FIG. 13A before the initiation of a 100 ms pulse burst, initiated upon detection of a P-wave. It is seen that the pulse burst did not induce additional atrial electrical activity. Whereas the R-R interval was essentially constant during the four cardiac cycles preceding stimulation, the R-R interval increased by over 50% in the first heartbeat following stimulation (i.e., t2>1.5t1), and was still elevated by over 20% in the second heartbeat following stimulation (i.e., t3>1.2*t1). It is additionally noted that the femoral arterial pressure (peak-to-peak time) also showed substantial lengthening, indicating that the stimulation provided in this experiment affected both the electrical and the mechanical behavior of the heart.

As can be observed in the graph, the stimulation had an effect on the next beat; not only did the next beat arrive after a longer than usual interval than in the preceding intervals, but the stimulation caused a steeper increase in femoral systolic blood pressure and was conducted through the His bundle in a different way from that seen in the preceding beats.

FIG. 13B is a graph showing data from an experiment performed in accordance with an embodiment of the present invention. The data shown is similar to that described hereinabove with reference to FIG. 13A, except that the PRI was set to 15 ms. In this experiment, the R-R interval increased by approximately 25% in the first heartbeat after stimulation.

FIG. 13C is a graph showing data from an experiment carried out using the asynchronous method described hereinabove, in accordance with an embodiment of the present invention. Pulse width was 0.01 ms, pulse amplitude was 2.4 V, and pulses were applied at 20 Hz, not synchronized to the cardiac cycle. Cardiac electrical and mechanical are seen to not be adversely affected by the stimulation.

FIG. 13D is a graph showing additional data from the experiment described hereinabove with reference to FIG. 13C, in accordance with an embodiment of the present invention. Approximately 15 seconds of baseline data are shown, in which no signal was applied to the heart. Then, at some point during the period marked “signal start,” the same signal as described with reference to FIG. 13C was applied to the SA node fat pad. After about 20 seconds of signal application, the signal was terminated, at the point marked “signal end.” FIG. 13D clearly shows the ability to apply a non-synchronized signal to the fat pads which substantially reduces heart rate, in accordance with an embodiment of the present invention.

FIG. 13E is a graph showing the results of an experiment carried out using the asynchronous method described hereinabove, to determine a therapeutic window which yields heart rate reduction, while avoiding atrial capture, in accordance with an embodiment of the present invention. In this experiment, for a range of pulse widths, signal voltage was increased until heart rate reduction was seen. This voltage was marked with a square. Signal voltage was increased further, until atrial capture was observed. This voltage is marked with a diamond. It is seen that for all of the pulse widths shown in FIG. 13E (0.01-0.08 ms), a window of at least a factor of two exists from the minimum voltage which yields heart rate reduction to the minimum voltage which yields atrial capture. Pulses were applied at 20 Hz, not synchronized to the cardiac cycle.

FIG. 13F is a graph showing the results of an experiment carried out using the asynchronous method described hereinabove, in accordance with an embodiment of the present invention. Pulses of 1.5 V and 20 Hz were applied over a range of pulse widths, from 0.01 to 0.05 ms. Heart rate reduction is seen to occur for pulse widths as low as 0.02 ms (HRR ˜7%), and to increase substantially as pulse width reaches 0.05 ms.

FIG. 13G is a graph showing the results of an experiment carried out using the asynchronous method described hereinabove, in accordance with an embodiment of the present invention. Pulses having a pulse width of 0.01 ms were applied at 20 Hz over a range of voltages. Heart rate reduction is seen for voltages as low as about 2.4 V, and the reduction increases to 40-50% for voltages of 5-6 V.

In an embodiment of the present invention, electrode assembly 22 comprises two electrode contacts configured to be placed in contact with the atrial wall in a vicinity of a parasympathetic epicardial fat pad. During an implantation procedure, control unit 24 separately drives each of the electrode contacts to apply stimulation to the wall, and determines respective heart-rate-lowering effects of the stimulation applied by the two electrode contacts. Whichever electrode contact has a great effect on heart rate is left in place, and the other electrode contact is repositioned at one or more addition locations. If stimulation at any of these other locations is found to have a greater heart-rate-lowering effect than at the location at which the first electrode contact remains, the other electrode contact is left at this new location, and the first electrode contact is repositioned at one or more locations. This testing and relocating is repeated until a satisfactory location has been identified, at which point the electrode contact positioned at this location is implanted in the wall. Alternatively, if the heart-rate-lowering effects of the two locations converge, either of the electrodes is implanted. Because an electrode contact is positioned at the identified location, there is no need to attempt to reposition an electrode contact at the location.

In an embodiment of the present invention, control unit 24 is configured to drive the electrodes to apply low-frequency bursts without synchronizing the bursts with any feature of the cardiac cycle of the subject. Typically, the frequency of the bursts is less than or equal to 2.5 Hz, e.g., less than or equal to 2 Hz (i.e., the number of bursts applied per second, not the number of pulses applied per second). Each of the bursts typically includes between 2 and about 20 pulses, with a pulse repetition interval (PRI) of between about 1 ms to about 30 ms, e.g., between about 3 and about 10 ms, such as about 5 ms. (The PRI is the time from the initiation of a pulse to the initiation of the following pulse within the same burst.) Using this technique, if the system should undesirably cause ventricular capture, the maximum ventricular rate would be no greater than the frequency of the burst. At such low frequencies, such unintended ventricular pacing would not be life-threatening. For some applications, such stimulation is applied when the subject is experiencing atrial fibrillation (AF), while for other applications, the stimulation is applied when the subject is not experiencing AF.

In an embodiment of the present invention, control unit 24 is configured to apply a signal to tissue in a vicinity of a fat pad, and to configure the signal to both pace the heart (i.e., cause capture) and activate parasympathetic tissue of the fat pad. Typically, an initial portion of the signal causes the pacing. For example, the signal may include bursts each of which include a plurality of pulses, and one or more of the initial pulses of the burst are configured to pace the heart. The control unit senses features of the cardiac cycle of the subject, and applies the signal at a desired portion of the cardiac cycle, as is known in the pacemaker art. Typically, the control unit senses whether the signal has caused capture, and increases the strength of the signal if it has not. At least the pulses configured to cause capture typically have an amplitude of at least 5 mA, and an aggregate duration of at least 2 ms. Typically, the signal is applied in the vicinity of the SA node fat pad; alternatively, the signal is applied in the vicinity of the AV node fat pad. For some applications, one or more electrode contacts are placed in the either the right or left atria.

In an embodiment of the present invention, control unit 24 is configured to use electrode contacts 30 and 32 to both apply fat pad stimulation and sense a local electrogram in the vicinity of the stimulation. The control unit measures a baseline electrogram before beginning application of the stimulation. If, during stimulation, the control unit detects a significant change in the sensed electrogram indicative of the undesired causing of capture by the stimulation, the control unit modifies one or more parameters of the stimulation to reduce the strength of the stimulation, or ceases stimulation.

In an embodiment of the present invention, to aid in the placement of the electrode contact, a CT scan is performed before the implantation, similar to the CT scan sometimes performed before AF ablation. Unlike such a conventional CT scan, in the present embodiment, the area of interest is the right atrium. Therefore, the time from injection of contrast material to triggering of the scan is shorter and the contrast material is less concentrated than conventionally applied for cardiac CT scans (conventional cardiac CT scans aim at the left side of the heart).

In an embodiment of the present invention, to aid in the placement of the electrode contact, prior to implantation a standard bipolar lead is used to find the location with the heart chamber at which application of stimulation causes the greatest heart-rate-lowering effect. The lead is placed at a plurality of locations, and stimulation is applied using the lead at each of the locations in order to determine at which location the stimulation causes the greatest heart-rate-lowering effect. The chronic implantable electrode contact is then positioned at the same location, e.g., using fluoroscopic guidance or a wireless position sensor. Alternatively, for some applications, the location of maximal heart rate reduction is found by applying test stimulation through the implantable electrode contact.

In an embodiment of the present invention, techniques are provided for avoiding inadvertent stimulation of the phrenic nerve. The right phrenic nerve is anatomically close to the SA node fat pad, and stimulation of the SA node fat pad might inadvertently stimulate the phrenic nerve under certain circumstances. To avoid such stimulation, possible stimulation of the phrenic nerve is noted during parameter setting (e.g., by noting irritation of the diaphragm), and the stimulation parameters are configured so as to not activate the phrenic nerve.

In an embodiment of the present invention, a bipolar electrode assembly is provided, comprising two monopolar electrode contacts. For some applications, the electrode assembly comprises more than two electrode contacts. For example, the use of more than two electrode contacts may compensate for post-implantation changes. For some applications, the control unit comprises multiple electrode contacts and switching capabilities, such that external programming can direct the stimulation current to different electrode contacts. For example, if an undesired reduction in stimulation efficacy is observed after the implantation, e.g., due to the development of local fibrosis, the stimulation can be directed through different electrode contacts.

In some embodiments of the present invention, an atrial electrode assembly is provided that hooks around the insertion of the superior vena cava into the right atrium.

In some embodiments of the present invention, the system comprises a first electrode contact, which is configured to be placed in the superior vena cava, and a second electrode contact, which is configured to be placed in the right atrium. For some applications, the control unit drives the first electrode contact to apply a cathodic current, and the second electrode contact to apply an anodal current, thereby limiting the potential of the stimulation to cause atrial depolarization, for example.

In some embodiments, the anode is larger than the cathode (e.g., in length and/or surface area) and/or segmented, such as to further reduce the likelihood of tissue depolarization in the vicinity of the anode.

In some embodiments of the present invention, the system comprises a first electrode contact, which is configured to be placed in the coronary sinus, and a second electrode contact, which is configured to be placed at an atrial site. For some applications, the control unit drives the first electrode contact to apply an anodal current, and the second electrode contact to apply a cathodic current, during the atrial refractory period. Alternatively or additionally, the control unit configures the first electrode contact to apply a cathodic current, and the second electrode contact to apply an anodal current, during the ventricular refractive period. In either case, the refractory periods may be absolute or relative refractory periods.

In an embodiment of the present invention, one or more of the electrode assemblies comprise an active fixation element, including an atrial-wall-penetrating screw-in fixation element that is configured to function as an electrode contact of the electrode assembly. In some embodiments the screw-in fixation element is placed in physical contact with the vagal ganglion plexus within the cardiac fat pads.

In an embodiment of the present invention, the electrode contacts are implanted in a chamber of the heart using a percutaneous approach.

In some embodiments of the present invention, a method for placing electrode contacts at an atrial site comprises testing placement of the electrode contacts by pacing the atrium while increasing vagal tone during a calibration stimulation period, which typically has a duration of between about 2 and about 15 seconds. The control unit paces and increases vagal tone by driving the electrode contacts to apply stimulation bursts that are shorter than the AERP at a rate that is above the basic normal sinus rhythm (NSR) rate, but not so rapid as to induce AF. For example, the rate may be between about 80 and about 140 bursts per minute, such as between about 90 to and about 130 bursts per minute. Upon conclusion of the calibration stimulation period, the atrium naturally returns to its original rate. However, because of the additional pulses applied during the AERP after capture has been achieved, pulses that may cause a vagomimetic effect if positioned correctly, the atrial rate falls below its original rate for several heartbeats, generally between about three and six beats. The control unit measures the R-R interval during at least one of these beats, e.g., during the one, two, or three of these beats. The degree of slowing detected is used to estimate the vagomimetic effect of applying stimulation at the site. If the achieved vagomimetic effect is insufficient, addition location(s) for the electrode contacts are tried until the desired effect is achieved. Alternatively or additionally, the method comprises applying the stimulation bursts as described, and observing the effect on pressure curves in the atria, ventricle, and/or pulmonary system.

Further alternatively or additionally, the method comprises applying stimulation bursts at a fixed rate (such as 120 per minute), with each burst having a duration that is shorter than the AERP, such as less than 90% of the AERP. When the stimulation is positioned at a site that elicits vagomimetic effects, the AERP shortens, resulting in double atrial excitation in each stimulation burst. Such shortening of the AERP can be observed from the atrial or ventricular electrogram.

Further additionally or alternatively, the method of placing the electrode contact includes applying stimulation bursts exclusively within the AERP, without causing atrial excitation. The natural sinus rate is then be observed for slowing that can verify the vagomimetic effect of the stimulation.

Further additionally or alternatively, a temporary pacing lead is positioned within the atria, to provide the atrial electrogram. This lead is removed once the correct position of the stimulating electrode contact is verified.

Further alternatively or additionally, the method comprises selecting a position of the electrode contacts responsively to subject-reported sensations, such as a feeling of warmth in the chest, a radiation of pain to the jaw or neck, or a burning sensation.

Further alternatively or additionally, the method of placing the electrode contact includes sensing the electrogram at the site and searching for irregularity in the ECG signal that is indicative for vagomimetic site. Such irregularity may be fractured ECG signal. Identify such irregularity may indicate that the electrode contact is positioned at a proper vagomimetic site.

Alternatively or additionally, the techniques described herein are used for classic pacing indications (e.g., bradycardia, sick sinus syndrome, or cardiac resynchronization), where instead of applying a single pacing signal, the device applies a burst of pulses, each burst having a duration that is shorter than the AERP, such as no more than 85% of the AERP. Typically the first pulse of each burst paces the atrium, and the subsequent pulses generate a vagomimetic response, but do not cause additional capture, because they are applied during the AERP.

“Heart failure,” as used in the specification and the claims, is to be understood to include all forms of heart failure, including ischemic heart failure, non-ischemic heart failure, and diastolic heart failure. A “screw,” as used in the present application, including in the claims, is to be understood broadly as including a screw, a corkscrew, or any helical element. “Chronically,” as used in the specification and in the claims, means for at least one month.

Techniques described herein for treating atrial fibrillation may also be performed for treating other forms of non-sinus atrial tachycardia, such as atrial flutter.

In some embodiments of the present invention, techniques and/or apparatus described in one or more of the following patents:
U.S. Pat. No. 6,006,134 to Hill et al.;
U.S. Pat. No. RE38,705 to Hill et al.; and/or
U.S. Pat. No. 6,292,695 to Webster, Jr. et al.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. patent application Ser. No. 10/205,474, filed Jul. 24, 2002, entitled, “Electrode assembly for nerve control,” which issued as U.S. Pat. No. 6,907,295
U.S. patent application Ser. No. 10/076,869, filed Feb. 15, 2002, entitled, “Low power consumption implantable pressure sensor,” which issued as U.S. Pat. No. 6,712,772
U.S. Provisional Patent Application 60/383,157 to Ayal et al., filed May 23, 2002, entitled, “Inverse recruitment for autonomic nerve systems”
U.S. patent application Ser. No. 10/205,475, filed Jul. 24, 2002, entitled, “Selective nerve fiber stimulation for treating heart conditions,” which published as US Patent Application Publication 2003/0045909
PCT Patent Application PCT/IL02/00068, filed Jan. 23, 2002, entitled, “Treatment of disorders by unidirectional nerve stimulation,” which published as PCT Publication WO 03/018113, and U.S. patent application Ser. No. 10/488,334, filed Feb. 27, 2004, in the US National Phase thereof
U.S. patent application Ser. No. 09/944,913, filed Aug. 31, 2001, entitled, “Treatment of disorders by unidirectional nerve stimulation,” which issued as U.S. Pat. No. 6,684,105
U.S. patent application Ser. No. 10/461,696, filed Jun. 13, 2003, entitled, “Vagal stimulation for anti-embolic therapy,” which issued as U.S. Pat. No. 7,321,793
PCT Patent Application PCT/IL03/00430, filed May 23, 2003, entitled, “Electrode assembly for nerve control,” which published as PCT Publication WO 03/099373
PCT Patent Application PCT/IL03/00431, filed May 23, 2003, entitled, “Selective nerve fiber stimulation for treating heart conditions,” which published as PCT Publication WO 03/099377
U.S. patent application Ser. No. 10/538,521, filed Jan. 11, 2006, entitled, “Efficient dynamic stimulation in implanted device,” which published as US Patent Application Publication 2006/0265027
U.S. patent application Ser. No. 10/719,659, filed Nov. 20, 2003, entitled, “Selective nerve fiber stimulation for treating heart conditions,” which published as US Patent Application Publication 2004/0193231
PCT Patent Application PCT/IL04/00440, filed May 23, 2004, entitled, “Selective nerve fiber stimulation for treating heart conditions,” which published as PCT Publication WO 04/103455
PCT Patent Application PCT/IL04/000496, filed Jun. 10, 2004, entitled, “Vagal stimulation for anti-embolic therapy,” which published as PCT Publication WO 04/110550, and U.S. patent application Ser. No. 10/560,654, filed May 1, 2006, in the US national stage thereof
U.S. patent application Ser. No. 10/866,601, filed Jun. 10, 2004, entitled, “Applications of vagal stimulation,” which published as US Patent Application Publication 2005/0065553
PCT Patent Application PCT/IL04/000495, filed Jun. 10, 2004, entitled, “Applications of vagal stimulation,” which published as PCT Publication WO 04/110549
U.S. patent application Ser. No. 11/022,011, filed Dec. 22, 2004, entitled, “Construction of electrode assembly for nerve control,” which published as US Patent Application Publication 2006/0136024
U.S. patent application Ser. No. 11/062,324, filed Feb. 18, 2005, entitled, “Techniques for applying, calibrating, and controlling nerve fiber stimulation,” which published as US Patent Application Publication 2005/0197675
U.S. patent application Ser. No. 11/064,446, filed Feb. 22, 2005, entitled, “Techniques for applying, configuring, and coordinating nerve fiber stimulation,” which published as US Patent Application Publication 2005/0267542
U.S. patent application Ser. No. 11/280,884, filed Nov. 15, 2005, entitled, “Techniques for nerve stimulation,” which published as US Patent Application Publication 2006/0106441
U.S. patent application Ser. No. 11/340,156, filed Jan. 25, 2006, entitled, “Method to enhance progenitor or genetically-modified cell therapy,” which published as US Patent Application Publication 2006/0167501
PCT Patent Application PCT/IL06/000616, filed May 25, 2006, entitled, “Suture loops for implantable device,” which published as PCT Publication WO 06/126201
U.S. patent application Ser. No. 11/359,266, filed Feb. 21, 2006, entitled, “Parasympathetic pacing therapy during and following a medical procedure, clinical trauma or pathology,” which published as US Patent Application Publication 2006/0206155
U.S. patent application Ser. No. 10/745,514, filed Dec. 29, 2003, entitled, “Nerve-branch-specific action-potential activation, inhibition, and monitoring,” which published as US Patent Application Publication 2005/0149154
U.S. patent application Ser. No. 11/234,877, filed Sep. 22, 2005, entitled, “Selective nerve fiber stimulation,” which published as US Patent Application Publication 2006/0100668
U.S. patent application Ser. No. 11/517,888, filed Sep. 7, 2006, entitled, “Techniques for reducing pain associated with nerve stimulation”
U.S. patent application Ser. No. 11/657,784, filed Jan. 24, 2007, entitled, “Techniques for prevention of atrial fibrillation,” which published as US Patent Application Publication 2007/0179543
U.S. patent application Ser. No. 11/724,899, filed Mar. 16, 2007, entitled, “Parasympathetic stimulation for termination of non-sinus atrial tachycardia”
U.S. Provisional Patent Application 60/937,351, filed Jun. 26, 2007, entitled, “Intra-atrial parasympathetic stimulation”
U.S. Provisional Patent Application 60/965,731, filed Aug. 21, 2007, entitled, “Intra-atrial parasympathetic stimulation”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
implanting in an atrial wall of a subject, from within an atrium, a first electrode contact in a vicinity of a parasympathetic epicardial fat pad of the subject;
implanting a second electrode contact in a body of the subject outside of a heart and a circulatory system; and
driving a current between the first and second electrode contacts, and configuring the current to cause parasympathetic activation of the fat pad.

2. The method according to claim 1, wherein implanting the first electrode contact comprises implanting, from within the atrium, a fixation element comprising a screw that comprises the first electrode contact.

3. The method according to claim 1, wherein implanting the second electrode comprises implanting the second electrode at a location that is not in physical contact with the heart or the fat pad.

4. The method according to claim 1, wherein configuring the current comprises configuring the current such that a pulse frequency, an amplitude, and a pulse width thereof have a product that is less than 12 Hz*mA*ms, and such that the current reduces a heart rate of the subject by at least 10% compared to a baseline heart rate of the subject in the absence of the application of the current.

5. The method according to claim 1, wherein implanting the second electrode contact comprises implanting the second electrode contact in a vicinity of left sides of right ribs of the subject.

6. The method according to claim 1, wherein implanting the second electrode contact comprises implanting the second electrode contact under right ribs of the subject.

7. The method according to claim 1, wherein implanting the second electrode contact comprises subcutaneously implanting the second electrode contact on a right side of a chest of the subject.

8. The method according to claim 1, wherein implanting the fixation element and the second electrode contact comprises implanting the fixation element and the second electrode contact such that a distance between the first and second electrode contacts is no more than 4 cm.

9. The method according to claim 1, wherein driving the current comprises configuring the current such that the first electrode contact serves as a cathode, and the second electrode contact as an anode.

10. The method according to claim 1, wherein implanting the second electrode element comprises implanting the second electrode element before implanting the fixation element, and wherein implanting the fixation element comprises:
positioning the first electrode contact at a plurality of locations of in the vicinity of the fat pad;
while the first electrode contact is positioned at each of the locations, driving the current between the first and second electrode contacts and sensing a vagomimetic effect; and
implanting the fixation element such that the first electrode contact is positioned at the one of the locations at which a greatest vagomimetic effect was sensed.

11-14. (canceled)

15. A method comprising:
implanting in an atrial wall of a subject, from within an atrium, a first electrode contact in a vicinity of a parasympathetic epicardial fat pad of the subject;
placing a second electrode contact within an organ of a circulatory system selected from the group consisting of: a superior vena cava, an inferior vena cava, a coronary sinus, a right pulmonary vein, a left pulmonary vein, and a right ventricular base; and
driving a current between the first and second electrode contacts, and configuring the current to cause parasympathetic activation of the fat pad.

16. The method according to claim 15, wherein implanting the first electrode contact comprises implanting, from within the atrium, a fixation element comprising a screw that comprises the first electrode contact.

17. The method according to claim 15, wherein configuring the current comprises configuring the current such that a pulse frequency, an amplitude, and a pulse width thereof have a product that is less than 12 Hz*mA*ms, and such that the current reduces a heart rate of the subject by at least 10% compared to a baseline heart rate of the subject in the absence of the application of the current.

18. The method according to claim 15, wherein the site includes the coronary sinus, wherein the fat pad includes an atrioventricular (AV) node fat pad, wherein placing comprises placing the second electrode contact in the coronary sinus, and wherein implanting comprises implanting the first electrode contact in the vicinity of the AV node fat pad.

19. The method according to claim 15, wherein implanting and placing comprise implanting the first electrode contact and placing the second electrode contact such that a distance between the first and second electrode contacts is no more than 2 cm.

20. The method according to claim 15, wherein driving the current comprises configuring the current such that the first electrode contact serves as a cathode, and the second electrode contact as an anode.

21-23. (canceled)

24. A method comprising:
implanting in an atrial wall of a subject, from within an atrium, at least two fixation elements comprising respective screws that comprise respective electrode contacts, such that the electrode contacts are positioned in a vicinity of a parasympathetic epicardial fat pad of the subject; and
driving a current between the electrode contacts, and configuring the current to cause parasympathetic activation of the fat pad.

25. The method according to claim 24, wherein implanting comprises implanting at least one of the fixation elements such that the electrode contact thereof is positioned entirely within the fat pad, and no other portion of the at least one of the fixation elements is in direct electrical contact with tissue of the atrial wall.

26. The method according to claim 24, wherein at least one of the screws has a proximal portion having a non-conductive external surface, and a distal portion having a conductive external surface that serves as the electrode contact of the screw.

27-112. (canceled)
